(19) United States
(12) Patent Application Publication
(10) Pub. No.: US 2020/0011885 A1
(43) Pub. Date: Jan. 9, 2020

(54) A CELL-BASED ASSAY AND KITS FOR ASSESSING SERUM ANTICHOLINERGIC ACTIVITY

(71) Applicant: Centre for Addiction and Mental Health, Toronto (CA)

(72) Inventors: Jose NOBREGA, Etobicoke (CA); Bruce POLLOCK, Toronto (CA); Roger RAYMOND, Toronto (CA)

(21) Appl. No.: 16/490,680

(22) PCT Filed: Mar. 2, 2018
(86) PCT No.: PCT/CA2018/050245

§ 371 Date: Sep. 3, 2019

Related U.S. Application Data
(60) Provisional application No. 62/466,651, filed on Mar. 3, 2017.

(51) Int. Cl. G01N 33/94; G01N 33/567; G01N 33/68

(52) U.S. Cl. CPC G01N 33/944; G01N 33/567; G01N 2800/2814; G01N 2333/726; G01N 33/6896

(57) ABSTRACT

Provided herein are methods for determining the level of muscarinic acetylcholine receptor subtype-(M1 receptor) anticholinergic activity in a blood serum sample, the method comprising the steps of removing protein from the blood serum sample by treatment with perchloric acid (PCA) to produce a PCA-treated serum sample; incubating the PCA-treated serum sample with a membrane preparation from cultured cells expressing the M1 receptor and an M1 receptor ligand; detecting an amount of binding of the M1 receptor ligand to the M1 receptor and comparing the amount of binding to a standard to determine the level of M1 receptor anticholinergic activity in the blood serum sample. Also provided are kits for performing the method.

### FIELD OF INVENTION

The present invention relates generally to the measurement of anticholinergic activity. More specifically, the present invention relates to methods and kits for assessing anticholinergic activity of a sample.

### BACKGROUND

Cholinergic receptor blockade in the central nervous system (CNS) is associated with impaired cognitive function and for this reason medications with anticholinergic activity are often carefully prescribed and dosed. However, many prescription and non-prescription drugs have varying degrees of anticholinergic activity, and when these drugs are combined, a significant amount of anticholinergic activity may result.⁵ The elderly, who very often take multiple medications for various different types of health issues, are particularly vulnerable in this respect particularly since CNS cholinergic function diminishes with aging. To assess the total burden of anticholinergic activity, a serum anticholinergic activity assay (SAA) was introduced in the early 1980s by Tune and Coyle^{1, 2} and has since been used as a putative marker of cognitive dysfunction in several conditions, albeit not always with consistent results³⁻¹². The original assay was based on the displacement of 3H-QNB binding to rat brain homogenates by anticholinergics in human serum. Subsequently, however, questions were raised concerning the basic validity of the SAA protocol and several potential limitations have been identified.⁹ Among these is a potential role for large serum proteins which may significantly mask or distort SAA values.¹³ Another issue refers to the fact that the original protocol did not discriminate between various subtypes of muscarinic receptors.¹⁴ This may be particularly relevant for studies correlating SAA with cognitive status, given that only two of the five known muscarinic receptor subtypes (M1 and M2) have been shown to be involved in cognitive functions.¹⁴ These and other issues have highlighted the need for an alternative protocol to assess total anticholinergic activity in human serum.

### SUMMARY OF THE INVENTION

According to an embodiment of the present invention there is provided a method for determining the level of muscarinic acetylcholine receptor subtype-1 (M1 receptor) anticholinergic activity in a blood serum sample, the method comprising:
removing protein from the blood serum sample by treatment with perchloric acid (PCA) to produce a PCA-treated serum sample;
incubating the PCA-treated serum sample with a membrane preparation from cultured cells expressing the M1 receptor and an M1 receptor ligand;
detecting an amount of binding of the M1 receptor ligand to the M1 receptor and comparing the amount of binding to a standard to determine the level of M1 receptor anticholinergic activity in the blood serum sample.

In a further embodiment, there is provided a method as described herein wherein the M1 receptor ligand is [3H] quinuclidinyl benzilate (3H-QNB), [3H] N-methyl-scopolamine (3H-NMS) or [3H] pirenzepine (3H-PZP). Any other isotopic label which permits efficient detection also may be used as may be any other suitable receptor ligand. In a preferred embodiment the M1 receptor ligand is 3H-QNB or 3H-NMS.

There is also provided a method as described herein, wherein the blood serum sample is derived from a patient or subject that exhibits one or more signs or symptoms of elevated M1 receptor anticholinergic activity, is suspected of having elevated blood levels of M1 receptor anticholinergic activity, exhibits no symptoms of elevated M1 receptor anticholinergic activity, or wherein the level of anticholinergic activity is unknown.

Also provided by the present invention is a method as described herein wherein the standard is atropine and wherein displacement by atropine of ligand binding to the M1 receptor is performed to generate one or more standard curves. The binding of atropine to the M1 receptor may be performed under essentially the same conditions as the M1 receptor binding to the M1 ligand. In an embodiment of the present invention, the level of M1 receptor anticholinergic activity may be expressed as atropine equivalents wherein the level of M1 receptor anticholinergic activity in the blood serum sample is calculated or estimated on the basis of the amount of atropine that would provide a substantially similar or identical degree of inhibition of the specific binding of the M1 receptor ligand to the M1 receptor.

In a further embodiment of the present invention, there is provided a method as described herein, wherein an elevated M1 receptor anticholinergic activity is equivalent to or higher than about 20, 25, 30, 35, 40, 45, 50, 55, 60, 65, 70, 75, 80, 85, 90, 95, 100, 105, 110, 115, 120, 125, 130, 135 or 140 pmol/mL atropine, and optionally associated with an age, a minimum age or an age range.

The present invention also contemplates a method as described herein wherein the M1 receptor is a rat receptor or human M1 receptor. Receptors from other species may be employed, particularly those which exhibit 100% identity to the rat or human M1 receptor.

In a further embodiment, there is provided a method as described herein which employs about 20 to 35 μg of membrane preparation, for example, but not limited to 20, 21, 22, 23, 24, 25, 26, 27, 28, 29, 30, 31, 32, 33, 34 or about 35 μg of membrane preparation. In a preferred embodiment about 27 μg of membrane preparation is employed in a 96 multiwell plate.

Also provided herein is a method wherein a volume of serum is deproteinized to perform the method. For example, but not to be considered limiting in any manner, about 1.5 mL of serum may be deproteinized and then an aliquot of that deproteinized solution, for example, but not limited to 100 μL may be employed in the method. The manufacturer of the protein deproteinization kit employed herein considers the deproteinized serum samples to be diluted to about 76% of the original concentration.

Also provided is a method as described herein wherein the ratio of the membrane preparation: treated serum sample is about 0.1 g:1 L to 0.4 g:1 L, for example, but not limited to about 0.1, 0.15, 0.2, 0.25, 0.3, 0.35 or 0.4 g:1 L. In a preferred embodiment, which is not meant to be limiting in any manner, the method employs about 100 μl deproteinized serum with about 27 μg M1WT3 protein. In a further embodiment the method employs about 10, 11, 12, 13, 14, 15, 16, 17, 18, 29, 20, 21, or 22 nM of ligand. In a preferred embodiment, the method employs about 16 nM of ligand which is close to the Kd of the ligand with the receptor, which is preferred. The nature of the ligand and its concentration may be changed or varied as needed or required as would be understood by a person of skill in the art.

In a further embodiment of the present invention, there is provided a method as described herein, wherein binding of the M1 receptor to M1 ligand employs a buffer comprising about 20 mM HEPES, about 100 mM NaCl and about 10 mM MgCl₂ adjusted to a pH of about 7.4. Other suitable buffers also may be used, for example, but not limited to about 50mM Na₂PO₄ pH 7.7 and about 10 mM KNaPO₄ pH 7.4.

Also provided is a method as described herein, wherein the PCA-treated serum sample, membrane preparation, M1 ligand and buffer are mixed at about 0° C. and the incubating step is performed at about 20 to 25 degrees, for example, but not limited to 24° C. for between about 30 and 120 minutes, for example, but not limited to 60 min.

In a further embodiment, after the step of incubating and before the step of detecting, an unbound M1 ligand is removed by filtering the membrane preparation on a filter with a pore size suitable for filtering unbound M1 ligand and retaining the M1 receptor followed by rinsing the membrane preparation.

Also provided herein is a method for assessing anticholinergic activity of a sample, the method comprising:
obtaining a membrane preparation from cultured cells expressing rat muscarinic receptor subtype 1 (M1 receptor);
incubating the treated serum sample with the membrane preparation and an M1 ligand;
detecting an amount of binding of the M1 ligand to the M1 receptor and comparing the amount of binding to a standard.

There is also provided a method for assessing anticholinergic activity of a serum sample, the method comprising:
removing protein from the serum sample by treatment with perchloric acid to produce a treated serum sample;
culturing cells expressing rat muscarinic receptor subtype 1 (M1 receptor);
obtaining a membrane preparation from the cells;
incubating the treated serum sample with the membrane preparation and an M1 ligand, wherein the M1 ligand is 3H-QNB or 3H-NMS;
removing unbound M1 ligand;
detecting an amount of binding of the M1 ligand to the M1 receptor and comparing the amount of binding to a standard.

In a further embodiment there is provided a method of modulating serum anticholinergic activity in a patient or subject about to receive medication or that is currently receiving medication, the method comprising:
obtaining a serum sample from the patient;
removing protein from the serum sample by treatment with perchloric acid to produce a treated serum sample;
obtaining a membrane preparation from cultured cells expressing muscarinic receptor subtype 1 (M1 receptor);
incubating the treated serum sample with the membrane preparation and an M1 ligand;
detecting an amount of binding of the M1 ligand to the M1 receptor; and quantifying the anticholinergic activity in the serum sample, and;
modulating the type, dosage, timing, dosage form, or delivery route of the medication, or any combination thereof.

Also provided is a method as described herein that further comprises repeating the steps before the modulating step to determine if the modulating changed the serum anticholinergic activity of the patient or subject. In a preferred embodiment, the modulating reduces serum anticholinergic activity of the patient or subject.

Also provided herein is a method of modulating serum anticholinergic activity of a patient receiving medication and exhibiting one or more signs of cognitive side effects, the method comprising:
obtaining a serum sample from the patient;
removing protein from the serum sample by treatment with perchloric acid to produce a treated serum sample;
obtaining a membrane preparation from cultured cells expressing muscarinic receptor subtype 1 (M1 receptor);
incubating the treated serum sample with the membrane preparation and an M1 ligand;
detecting an amount of binding of the M1 ligand to the M1 receptor and comparing the amount of binding to a standard; and modulating the type, dosage, timing, dosage form, or delivery route of the medication, or any combination thereof.

In a further embodiment, there is provided a method that further comprises repeating the steps before the modulating step to determine if the modulating decreased the serum anticholinergic activity.

The present invention also provides a kit for assessing or determining anticholinergic activity comprising one or more of the following components in any combination: cells expressing an M1 receptor, one or more cell culture media, one or more cell wash media, one or more buffers, protein concentration assay determination reagent(s), one or more anticholinergic compounds or compositions, atropine, one or more multi-well plates, M1 receptor membrane preparations adhered to a plate, filter or other substrate, one or more filtration membranes, scintillation fluid, one or more M1 ligands, deproteinization solution, perchloric acid, perchloric acid neutralization solution, data analysis software, serum containing one or more anticholinergic compounds or compositions, glassware, centrifuge tubes, instructions for performing the anticholinergic assay or any combination thereof.

In a preferred embodiment, which is not meant to be limiting in any manner, the kit comprises:
perchloric acid to remove protein from the serum sample to produce a treated serum sample;
perchloric acid neutralizing solution;
a membrane preparation from cultured cells expressing muscarinic receptor subtype 1 (M1 receptor); and
an M1 ligand.

Optionally the kit as described herein may further comprise one or more multiwell plates, one or more multi-well plates comprising a filter with a pore size suitable for filtering unbound M1 ligand and retaining the M1 receptor, buffer, scintillation fluid or any combination thereof.

In still a further embodiment there is provided a kit for assessing anticholinergic activity of a serum sample, the kit comprising:
perchloric acid to remove protein from the serum sample to produce a treated serum sample;
an M1 ligand;
buffer;
   one or more multiwell plates comprising, in each well, a membrane preparation from cultured cells expressing rat muscarinic receptor subtype 1 (M1 receptor), or one or more multiwell plates comprising a filter with a pore size capable of filtering unbound M1 ligand and retaining the M1 receptor. The membrane preparation expressing rat muscarinic subtype 1 (M1 receptor) may be bound to the filter through which unbound M1 ligand in the assay may be washed away, or it may be provided separately.

In another embodiment, there is provided herein a method for identifying a subject as being at risk of having or developing cognitive impairment, the method comprising:
providing a blood serum sample from the subject;
removing protein from the blood serum sample by treatment with perchloric acid (PCA) to produce a PCA-treated serum sample;
incubating the PCA-treated serum sample with a membrane preparation from cultured cells expressing the M1 receptor and an M1 receptor ligand; and
detecting an amount of binding of the M1 receptor ligand to the M1 receptor and comparing the amount of binding to a standard to determine a level of muscarinic acetylcholine receptor subtype-1 (M1 receptor) anticholinergic activity in the blood serum sample;
wherein an elevated level of M1 anticholinergic activity in the blood serum sample as compared to a healthy control level identifies the subject as being at risk of having or developing cognitive impairment.

In yet another embodiment, the method may further comprise a step of:
subjecting the subject identified as being at risk of having or developing cognitive impairment to a Cambridge Neuropsychological Test Automated Battery (CANTAB-AD) to further assess cognitive impairment of the subject.

In still another embodiment, the subject may be a subject being treated with at least one drug having anticholinergic properties.

In yet another embodiment, the cognitive impairment may be in the spatial working memory cognitive domain.

As will be understood, while the above embodiments pertain to determining the level of muscarinic acetylcholine receptor subtype-1 (M1 receptor) anticholinergic activity in a blood serum sample, similar methods may be employed to determine the level of other muscarinic receptor anticholinergic activity in a sample using a suitable receptor ligand and suitable membrane preparation expressing the other muscarinic receptor.

### BRIEF DESCRIPTION OF THE DRAWINGS

FIG. 1. [3H] QNB, [3H] NMS and [3H] PZP binding in M1WT3 cells. (A-C): Saturation curves for each radioligand. Each point represents specific binding defined in the presence of 10 μM atropine, done in triplicate. Note the overall similarity between [3H] QNB and [3H] NMS (A and B), while [3H] PZP (C) required much higher concentrations to achieve saturation. (D-F): Atropine displacement of 0.16 nM [3H] QNB (D), [3H] NMS (E) and [3H] PZP (F).

FIG. 2: Detection of anticholinergic activity in spiked serum. Normal human serum was spiked with a test solution consisting of a mixture of different amounts of various medications known to possess anticholinergic activity (A and B), as will be described below. The spiked serum solution was then diluted with normal unspiked serum to achieve different concentrations of anticholinergic activity, from 0% dilution (original spiked serum) to 100% dilution (normal drug-free serum). Values are means±SEM, expressed as atropine equivalents. N=6 per point.

FIG. 3: Direct comparison of [3H] QNB (A) and [3H] NMS (B) 0.12 nM in M1WT3 cells vs. rat brain tissue. Significant differences are seen except at the lowest drug concentrations. N=6 per point. *** P<0.002 Sidak-adjusted comparisons following a significant Dilution X Type of Assay ANOVA interaction (p<0.001).

FIG. 4. Effects of filtration and protein extraction on SAA values obtained with [3H] QNB (A) and [3H] NMS (B). *** P<0.002, ** p<0.02 compared to corresponding unextracted point, Sidak-adjusted comparisons following a significant Dilution X Treatment interaction in ANOVA (P<0.001). N=6 per point in extracted and PCA-extracted groups, N=4 per point for YM-50 filtered group.

FIG. 5: Effects of normal serum on 3H-radioligand binding. In the absence of any exogenous anticholinergic compounds normal serum alone reduced binding by approximately 16% as compared to buffer (*** P<0.001, Sidak-adjusted test following a significant Medium X Treatment ANOVA interaction). Pretreatment of normal buffer with perchloric acid (PCA) restored binding to normal buffer levels. N=4 per group;

FIG. 6: Depicts a schematic of the study design for Example 2;

FIG. 7: SAA values before and after acute scopolamine (0.4 mg i.v.). (a) and (b): Individual data for all participants. Note that for each subject, each point represents the mean of 4 determinations. Standard errors are omitted for clarity. (c): 3H-QNB standard curve against atropine for calculating sample SAA values in atropine equivalents;

FIG. 8: CANTAB cognitive test scores pre (blue, solid bars) and post (red, striped bars) scopolamine. Each bar is the mean (and sem) for pre vs. post scopolamine for each variable. x-axis labels in (a)-(f) are abbreviations corresponding to variable headings provided in Table 1 which describes each of the variables, and Table 2 provides data in tabulated format. * p<0.05, ** p<0.025, *** p<0.012 paired t tests; and

FIG. 9: Correlation between SAA change and changes in CANTAB cognition measures. This figure provides an example of correlations between changes in SAA and changes in CANTAB measures (working memory).

### DETAILED DESCRIPTION

Described herein are embodiments illustrative of compositions, kits and methods for assessing anticholinergic activity of a sample. It will be appreciated that the embodiments and examples described herein are for illustrative purposes intended for those skilled in the art and are not meant to be limiting in any way. All references to embodiments or examples throughout the disclosure should be considered a reference to an illustrative and non-limiting embodiment or an illustrative and non-limiting example.

In the methods recited herein, assessing anticholinergic activity of a serum sample involves assessing the capacity of drugs and/or other compounds in the serum to bind to muscarinic receptors thereby reducing the binding of a test radioligand to, for example, but not limited to, the muscarinic M1 receptor. The procedure thus determines the total burden of anticholinergic activity at the receptor, irrespective of the type and amounts of individual anticholinergic compounds that may be present in serum. This is especially important when the serum may contain a combination of drugs and/or compounds that can result in high or elevated anticholinergic activity, possibly leading to negative cognitive or other side effects. The method is based on the competitive binding between specific ligands and anticholinergic drugs and/or compounds in the blood serum that interact with the muscarinic receptors, for example, but not limited to the M1 receptor. In an embodiment, which is not meant to be limiting in any manner, binding of the M1 ligand to the M1 receptor is reduced in a proportion to the concentration and potency of the anticholinergic drugs and/or compounds in the serum.

According to an embodiment of the present invention, there is provided a method for determining the level of muscarinic acetylcholine receptor subtype-1 (M1 receptor) anticholinergic activity in a blood serum sample, the method comprising:
removing protein from the blood serum sample by treatment with perchloric acid (PCA) to produce a PCA-treated serum sample;
incubating the PCA-treated serum sample with a membrane preparation from cultured cells expressing the M1 receptor and an M1 receptor ligand;
detecting an amount of binding of the M1 receptor ligand to the M1 receptor and comparing the amount of binding to a standard to determine the level of M1 receptor anticholinergic activity in the blood serum sample.

According to an alternate embodiment of the present invention, there is provided a method for assessing anticholinergic activity of a serum sample, the method comprising:
removing protein from the serum sample by treatment with perchloric acid to produce a treated serum sample;
obtaining a membrane preparation from cultured cells expressing rat muscarinic receptor subtype 1 (M1 receptor);
incubating the treated serum sample with the membrane preparation and an M1 ligand;
detecting an amount of binding of the M1 ligand to the M1 receptor and comparing the amount of binding to a standard.

In the context of the present invention, the term “membrane preparation” is meant to encompass any suitable cellular preparation that comprises membrane proteins, including the muscarinic receptors, for example, but not limited to the M1 muscarinic receptor. Any suitable technique known in the art may be utilized to obtain the membrane preparation. Particularly suitable techniques substantially maintain the structure and/or activity of the receptor as would be appreciated by a person of skill in the art. For example, and without being limiting, the membrane preparation may be obtained by a modified method of Lazareno et al.¹⁵ Briefly, cells may be homogenized on ice for 30 seconds, centrifuged at 40,000×g for 90 min at 4° C., rinsed with a solution comprising 20 mM HEPES and 0.1 mM EDTA adjusted to pH 7.4, centrifuged again at 40,000×g for 10 min, reconstituted, and stored at −80° C. Other suitable procedures known in the art may also be used to obtain the membrane preparation.

The membrane preparation may be obtained from cultured cells that express the M1 receptor. To obtain cultured cells that express the M1 receptor, molecular biology techniques commonly known in the field may be used. For example, an appropriate cell may be transformed with a vector comprising the necessary nucleic acid information to result in the expression of the M1 receptor. To confirm the presence of the M1 receptor on the transformed cells, the M1 receptor may be detected with the use of antibodies, or any other biochemical technique known in the art. In an embodiment of the present invention, ATCC® M1WT3 cells, which express rat muscarinic receptor subtype 1, may be used. In an embodiment of the present invention the M1 receptor is a rat M1 receptor. In a further embodiment, the M1 receptor is a human M1 receptor. In a further embodiment the cultured cells and the M1 receptor are from different species. In a further embodiment, the cultured cells and the M1 receptor are from the same species. In an embodiment the M1 receptor is a rat M1 receptor and the cultured cells are CHO cells.

The use of cell lines has distinct advantages over the use of rat brain tissue. Cells can be commercially purchased and grown in the laboratory as needed, thereby obviating the need to sacrifice experimental animals for the assays. Tests suggest that with the M1WT3 cell line, there is no appreciable loss of binding after 60 cycles of cell growth. In addition, the fact that much smaller quantities of material are needed makes it possible to achieve large-scale simultaneous processing, thereby increasing precision and reliability. A related advantage is the reduction in the total amount of M1 ligand needed per assay, which contributes significantly to reducing overall costs.

The removal of proteins, for example, large proteins, from serum may aid in removing interference caused by proteins in the method. Perchloric acid (PCL) deproteinization is one method that may be used in the removal of serum proteins. Briefly, the serum sample may be mixed with ice-cold PCA, incubated on ice for about 5 minutes and then centrifuged at about 13,000×g for 2 min. The supernatant may be removed and neutralized, after which the precipitated PCA may be removed to produce the treated serum sample.

After incubating the serum with the M1 ligand in the presence of the membrane preparation, the amount of binding of the M1 ligand to the M1 receptor is detected and quantified. Detection can be performed using any technology known in the art. For example, the M1 ligand may be a radioligand or may be labeled with a fluorescent dye, moiety or group where the radioactivity or fluorescence of the bound M1 ligand may be detected. Alternatively, the M1 ligand bound to the M1 receptor may be detected with the use of immunolabeling. In a preferred embodiment of the present invention, the M1 ligand is a radioligand. In a further embodiment of the present invention, the M1 ligand is [³H] quinuclidinyl benzilate ([3H] QNB), [³H] N-methyl-scopolamine ([3H] NMS) or [³H] pirenzepine ([3H] PZP). In a preferred embodiment, the M1 ligand is [3H] QNB or [3H] NMS.

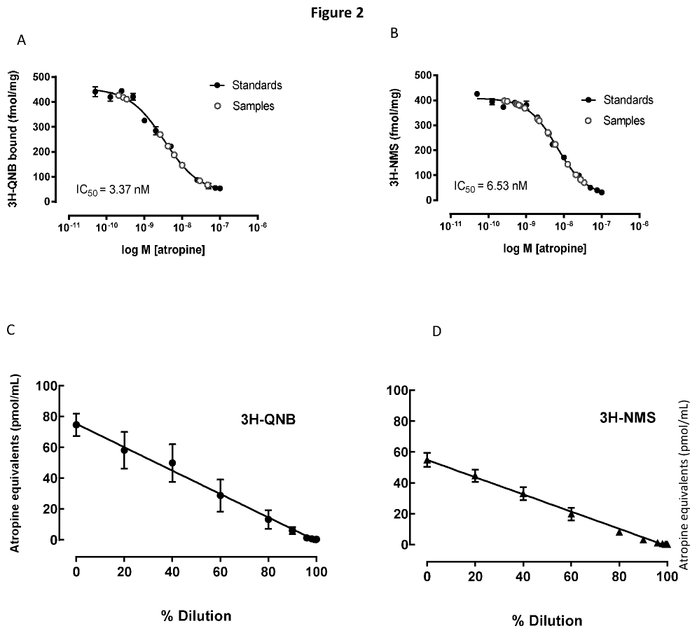

The binding of the M1 ligand to the M1 receptor is usually compared to a standard or reference. For example, one or more standard curves for the displacement of increasing concentrations of a particular anticholinergic drug, for example and without being limiting, atropine, may be performed with one or more concentrations of the M1 ligand that is used in the method. A standard curve may be constructed by plotting the reduction of M1 binding induced by increasing amounts of atropine. A subject's SAA level is estimated by the reduction that serum from this subject induces in M1 binding, and it is expressed in terms of the amount of atropine that would be necessary to achieve the same effect. The result may be expressed as atropine equivalents. The standard curves may be performed with concentrations of atropine ranging from, for example, but not limited to 0.0 nmol/mL to 100 nmol/mL, or more in serum. The results obtained from the method may be calculated on the basis of the amount of atropine (atropine equivalent in pmol/mL, or any other suitable units value) that would provide a similar or identical degree of inhibition. Examples of standard curves are shown in FIGS. 2A and 2B, with interpolated samples shown as open circles The level of anticholinergic activity may be expressed in relation to any standard; however, serum anticholinergic activity (SAA) results have traditionally been expressed as atropine equivalents ^{1,2}.

In an embodiment of the present invention, the blood serum sample may be derived from a patient or subject that exhibits one or more signs or symptoms of high or elevated M1 receptor anticholinergic activity, is suspected of having high or elevated blood levels of M1 receptor anticholinergic activity, exhibits no symptoms of high or elevated M1 receptor anticholinergic activity, or wherein the level of anticholinergic activity is unknown.

By the term “high” or “elevated” M1 receptor activity, it is meant an anticholinergic activity that is equal to or greater than a specific activity level, which may be recited in terms of pmol/mL atropine or any other appropriate unit as would be known in the art, for example, but not limited to 50, 55, 60, 65, 70, 75, 80, 85 90, 95, 100, 105, 110, 115, 120, 130, 135, 140 pmol/mL atropine or higher. Further the term may be defined by a range of any two values recited or any two values recited therein between. Signs or symptoms of elevated anticholinergic activity may include one or more cognitive side effects or non-cognitive side effects such as, but not limited to dementia, memory loss, cognitive decline, decrease in global cognitive functioning, psychomotor speed, decrease in visual and/or declarative memory, implicit learning or communication ability, confusion, disorientation, agitation, euphoria or dysphoria, respiratory depression, inability to concentrate, inability to sustain a train of thought, incoherent speech, irritability, wakeful myoclonic jerking, unusual sensitivity to sudden sounds, illogical thinking, photophobia, visual disturbances, visual, auditory, or other sensory hallucinations, orthostatic hypotension, urinary problems and/or kidney failure, salivary problems such as dry mouth, blurred vision, constipation, hypohydrosis, dizziness and the like. In a preferred embodiment, the signs include one or more cognitive side effects. In a further embodiment, the signs include one or more non-cognitive side effects.

In an embodiment of the method, after the step of incubating and before the step of detecting, unbound M1 ligand is removed, for example, but not limited to filtering the membrane preparation on a filter with a pore size suitable for removing unbound M1 ligand and retaining the M1 receptor/M1 ligand complex, followed by rinsing the membrane preparation. For example, but without wishing to be limiting, a GF/B filter, which has a pore size of about 1 μm, that is presoaked in 0.1% poly(ethyleneimine) may be used. The membrane preparation may be rinsed with any suitable physiological buffer, for example, a buffer comprising about 50 mM Tris HCl, about 150 mM NaCl adjusted to a pH of about 7.4 at about 0° C. Other buffers may be employed under other conditions as would be understood by a person of skill in the art.

It is contemplated that the method or methods of the present invention can be performed in a multiwell plate, for example, but not limited to a plate comprising 6, 24, or 96 wells. Plates comprising other numbers of wells also may be used. Similarly other suitable devices and/or systems may be employed as would be understood by a person of skill in the art.

The present invention also provides for a method for assessing anticholinergic activity of a serum sample, the method comprising:
removing protein from the serum sample by treatment with perchloric acid to produce a treated serum sample;
culturing cells expressing rat muscarinic receptor subtype 1 (M1 receptor);
obtaining a membrane preparation from the cells;
incubating the treated serum sample with the membrane preparation and an M1 ligand, wherein the M1 ligand is 3H-QNB or 3H-NMS;
removing an unbound M1 ligand;
detecting an amount of binding of the M1 ligand to the M1 receptor and comparing the amount of binding to a standard.

In some instances, it may be desirable to test samples other than serum samples to assess anticholinergic activity. Such a sample may comprise compounds, compositions, drugs or medicaments which exhibit anticholinergic activity dissolved in a solvent such as, but not limited to water or the like. In such cases, removing protein from samples may not be required, particularly if it is known in advance that the samples do not comprise protein or other components which should be removed by deproteinization. Thus, according to a further embodiment of the present invention, there is provided a method for assessing anticholinergic activity of a sample, the method comprising:
incubating the sample with a membrane preparation comprising muscarinic receptor subtype 1 (M1 receptor) and an M1 ligand;
detecting an amount of binding of the M1 ligand to the M1 receptor and comparing the amount of binding to a standard.

The methods presented herein are useful for determining, assessing and understanding the level of anticholinergic components in a sample such as a patient's blood sample. This in turn may assist in determining, assessing and/or understanding the effects of one or more medications administered to a patient on serum anticholinergic activity, allowing for the ability to modulate the type of medication(s), dosage, dosage form, delivery route and/or dosage regimen of medication administered to the patient depending on the result.

Therefore, in one embodiment, the present invention provides for a method of modulating serum anticholinergic activity in a patient receiving medication, the method comprising:
obtaining a serum sample from the patient;
removing protein from the serum sample by treatment with perchloric acid to produce a treated serum sample;
obtaining a membrane preparation from cultured cells expressing rat muscarinic receptor subtype 1 (M1 receptor);
incubating the treated serum sample with the membrane preparation and an M1 ligand;
detecting an amount of binding of the M1 ligand to the M1 receptor and comparing the amount of binding to a standard; and
modulating the dosage and/or type of the medication.

Modulating the type of medication may encompass stopping administration of a medication, changing a medication, adding one or more new medications or any combination thereof. Modulating the dosage of medication may comprise lowering or increasing the dosage of a medication as desired and/or necessary. Further, the dosage form or delivery route of a medication or combination of medications may be changed depending on the result of the assay. For example, but without wishing to be limiting in any manner, it may be desirable to change medications from a quick release dosage form to a sustained delivery dosage form. Similarly it may be desirable to change from oral delivery to intravenous delivery of the medicament. Further, it may be desirable to change from a once a day delivery regimen to smaller doses multiple times a day, for example.

The present invention further contemplates assessing the serum anticholinergic activity after the medication has been modulated to determine if the change in the medication had the intended outcome.

The present invention also provides for a method of modulating serum anticholinergic activity of a patient receiving medication and exhibiting one or more cognitive side effects, the method comprising:
obtaining a serum sample from the patient;
removing protein from the serum sample by treatment with perchloric acid to produce a treated serum sample;
obtaining a membrane preparation from cultured cells expressing rat muscarinic receptor subtype 1 (M1 receptor);
incubating the treated serum sample with the membrane preparation and an M1 ligand;
detecting an amount of binding of the M1 ligand to the M1 receptor and comparing the amount of binding to a standard; and
modulating the dosage and/or type of the medication to decrease the serum anticholinergic activity.

The methods described herein also may comprise a step of monitoring a subject for one or more signs or symptoms of high or elevated serum anticholinergic activity. Signs or symptoms of high anticholinergic activity include, may include, without limitation a variety of cognitive side effects.

The present invention further contemplates assessing the serum anticholinergic activity after the medication has been modulated to determine if the change in the medication decreased serum anticholinergic activity, thereby possibly decreasing the cognitive side effects.

The present invention also provides for a kit for assessing anticholinergic activity of a serum sample, the kit comprising:
a membrane preparation from cultured cells expressing the muscarinic receptor subtype 1 (M1 receptor); and
an M1 ligand.

The kit may further comprise a perchloric acid solution to remove protein from the serum sample to produce a treated serum sample, perchloric acid neutralization solution, one or more multi-well plates, one or more multiwell plate comprising a filter with a pore size suitable for removing unbound M1 ligand while retaining the M1 receptor-M1 ligand complex intact, one or more buffers, cell culture medium, scintillation fluid, instructions for assessing anticholinergic activity in a sample or any combination thereof.

In a further embodiment, the kit further comprises one or more multiwell plates, or one or more multiwell plates comprising a filter with a pore size suitable of filtering unbound M1 ligand and retaining the M1 receptor, and buffer. In an even further embodiment, the kit further comprises scintillation fluid.

Another embodiment of a kit of the present invention comprises:
perchloric acid to remove protein from the serum sample to produce a treated serum sample;
an M1 ligand;
buffer;
one or more multi-well plate comprising, in each well, a membrane preparation from cultured cells expressing rat muscarinic receptor subtype 1 (M1 receptor), or one or more multiwell plates comprising a filter with a pore size suitable for filtering unbound M1 ligand while retaining the M1 receptor-M1-ligand complex, or a combination thereof.

In a further embodiment there is also contemplated a method as described above and herein throughout, wherein the muscarinic acetylcholine receptor is the M2 receptor or both the M1 and M2 receptors. Preferably the M1 or M2 or both M1 and M2 receptors are expressed on the surface of cultured cells and maintain native or near native conformation and/or activity as compared to the same receptor(s) in their natural environment. In still a further embodiment, the cultured cells express one or more other muscarinic acetylcholine receptors, such as, but not limited to the M3, M4 or M5 receptors, alone or in combination with the M1 receptor, M2 receptor or both M1 and M2 receptors and preferably under the same conditions as described above and herein throughout. Also contemplated herein are methods which separately employ a cell line expressing the M1 receptor and separate cell lines expressing individual muscarinic receptors. Such methods may be preferred in situations to determine anticholinergic activity in samples which may be relevant to multiple muscarinic receptor types, for example, but not limited to M1 and M2; M1, M2 and M3; M1 and M4; M1 and M5, M1, M4 and M5; M1, M2, M3, M4, and M5, and the like. In an embodiment wherein the method employs cells expressing the M2/M4 receptors, the ligand 3H-AFDX-384 may be employed. In an embodiment wherein the method employs cells expressing the M1/M3 receptors, the radioligand 3H-4-DAMP may be employed. Other suitable ligands may be used for other receptors as would be understood by a person of skill in the art.

The method described herein provides improvements over the original SAA assay described by Tune and Coyle.^{1,2} The use of cultured cells expressing the M1 receptor offers advantages over the use of rat brain tissue as the latter rat brain tissue expresses several types of muscarinic receptors, all of which may not be involved in human cognitive function. Further, the use of cultured cells, compared to rat brain cells, reduces costs, provides ease of maintenance and obviates the need to sacrifice animals. In addition, more precise results may be obtained by PCA pre-treatment to neutralize potential effects of endogenous proteins in serum samples. The method described herein addresses defects in the prior art to assaying serum anticholinergic activity, for example, but not limited to requiring less sample volumes, requiring lesser amounts of M1 ligand and obviating the need to use animal brain tissue.

In still another embodiment, there is provided herein a method for identifying a subject as being at risk of having or developing cognitive impairment, the method comprising:
providing a blood serum sample from the subject;
removing protein from the blood serum sample by treatment with perchloric acid (PCA) to produce a PCA-treated serum sample;
incubating the PCA-treated serum sample with a membrane preparation from cultured cells expressing the M1 receptor and an M1 receptor ligand; and
detecting an amount of binding of the M1 receptor ligand to the M1 receptor and comparing the amount of binding to a standard to determine a level of muscarinic acetylcholine receptor subtype-1 (M1 receptor) anticholinergic activity in the blood serum sample;
wherein an elevated level of M1 anticholinergic activity in the blood serum sample as compared to a healthy control level identifies the subject as being at risk of having or developing cognitive impairment.

In another embodiment, the subject may be an aged or elderly subject. In certain embodiments, for example, the subject may be an older subject, such as a subject between about 59-86 years old.

As will be understood, the healthy control level may be any suitable reference level (i.e. a threshold, or range) determined as being indicative of a healthy state in which no significant cognitive impairment is experienced. By way of example, the healthy control level may be a reference level determined for an aged or older control group of subjects being cognitively intact with good medical health. In certain embodiments, the control group may comprise subjects of about 59-86 years old, for example. In certain embodiments, the healthy control level may be a reference level determined as being indicative of a healthy state in which no significant cognitive impairment is experienced, as indicated by a MoCA result of higher than about 24, and a score of about 0 in the CDR. In certain embodiments, the healthy control level may be a pre-determined level derived by performing the method on a group of healthy non-cognitively impaired control subjects. In certain embodiments, the healthy control level may be a level previously measured in the subject while the subject was not cognitively impaired, or may be a level previously measured in the subject prior to treating the subject with a drug with anticholinergic properties, for example.

In yet another embodiment, the method may further comprise a step of:
subjecting the subject identified as being at risk of having or developing cognitive impairment to a Cambridge Neuropsychological Test Automated Battery (CANTAB-AD) to further assess cognitive impairment of the subject.

In still another embodiment, the subject may be a subject being treated with at least one drug having anticholinergic properties.

In yet another embodiment, the cognitive impairment may be in the spatial working memory cognitive domain.

### EXAMPLES

### Example 1

### Analysis of Anticholinergic Activity Levels of Blood Serum Samples

### 1. Cell Culture and Obtaining Membrane Preparations

Chinese hamster ovary (CHO) cells stably expressing rat M1 muscarinic receptors (M1WT3; American Type Culture Collection, ATCC, Manassas, Va.) were grown to 90% confluence in 25 mL F-12K medium (ATCC) supplemented with 10% FBS, 100 units/mL penicillin, 100 μg/mL streptomycin and 100 μg/mL geneticin in a T175 flask at 37° C. in humidified air and 5% CO2. Cells were harvested using 8 mL Accutase® (Sigma-Aldrich, Oakville, ON), rinsed with magnesium- and calcium-free Dulbecco's PBS, and stored at −80° C. in 20 mM HEPES, 10 mM EDTA, pH 7.4. Membrane preparations were obtained according to Lazareno et al,¹⁵ with minor modifications. Briefly, cells were homogenized for 30 seconds on ice with a polytron (Brinkmann, Canada), centrifuged at 40,000×g for 90 min at 4° C., rinsed with 20 mM HEPES, 0.1 mM EDTA pH 7.4, centrifuged again at 40,000×g for 10 min, reconstituted, and stored at −80° C. Protein concentration was determined using the Pierce BCA Protein Assay Kit (Thermo Fisher Scientific, Nepean, ON).

### 2. Serum Test Solutions

To ascertain the effects of anticholinergics in serum and to simulate patient samples containing varying levels of anticholinergic medications, normal human serum (Millipore, Billerica, Mass.) was spiked with a test solution containing known amounts of compounds with varying degrees of anticholinergic activity⁵. The test serum solution consisted of: Clozapine 1000 ng/ml, Amitriptyline 100 ng/ml, Scopolamine 0.05 ng/ml, Chlorpromazine 50 ng/ml, and Paroxetine 100 ng/ml. The test serum solution was then diluted with normal serum in 20% steps from 100% down to 0% in order to obtain varying amounts of anticholinergic drugs in serum samples.

### 3. Binding Assay Protocol in M1WT3 Cells

The binding assay consisted of 100 μL of serum, 27 μg of M1WT3 protein and nanomolar concentrations of the appropriate M1 ligand in buffer (20 mM HEPES, 100 mM NaCl and 10 mM MgCl2, pH 7.4) in a total volume of 500 μL, assembled on ice in a 96-well Whatman™ uniplate (GE Healthcare, Mississauga, ON). Receptor concentration was estimated to be approximately 713 fmol/mg membrane protein. The microplate was then incubated at 24° C. for 60 min. In all cases nonspecific binding was defined in the presence of 10 μM atropine. Membranes were collected by filtration on GF/B filtermats presoaked in 0.1% PEI, using a Filtermate Harvester (Perkin Elmer, Waltham, Mass.), then rinsed with 50 mM Tris HCl, 150 mM NaCl pH 7.4 at 0° C. Filtermats were dried, soaked in scintillation fluid (Microscint-PS, Perkin Elmer) and sealed. Bound radioactivity was counted in a Microbeta-2 microplate scintillation counter (Perkin Elmer).

### 4. Deproteinization

### 4.1 Protein Filtration Experiments

Filtration experiments to remove large proteins from serum were performed with 50 kDa membranes (YM-50, Microcon, Bedford, Mass.), as described by Cox et al.¹³

### 4.2 Protein Removal by Precipitation

Perchloric acid deproteinization experiments were performed with a deproteinizing kit (catalog #K808-200; BioVision, Milpitas, Calif.) according to manufacturer's instructions. Briefly, 400 μL of sample were mixed with 100 μL of ice-cold PCA, placed on ice for 5 min and the centrifuged at 13,000×g for 2 min; 380 μL of the supernatant was transferred to a fresh tube to which was added 20 μL of ice-cold Neutralization Solution. The precipitate was resuspended and mixed to neutralize the sample and precipitate excess PCA. Samples were placed on ice for 5 min and spun briefly prior to use.

### 5. M1 Ligands

[3H] quinuclidinyl benzilate ([3H] QNB, 30-60 Ci/mmol), [3H] N-methyl-scopolamine ([3H] NMS, 70-90 Ci/mmol) and [3H] pirenzepine ([3H] PZP, 70-90 Ci/mmol) were obtained from PerkinElmer (Waltham, Mass.).

### 6. Binding Assay in Rat Brain Homogenates

Binding of [3H] QNB to rat brain homogenates followed established protocols (Cox et al., 2009). Briefly, 27 μg of rat brain tissue (frontal cortex and caudate-putamen) was used in a total volume of 500 μL, consisting of phosphate-buffered saline, 0.16 nM [3H] QNB and 20 μL of the spiked serum test solution diluted to various concentrations as described above.

### 7. SAA Assays

Standard curves were constructed by measuring the displacement of radioligand binding by 13 different concentrations of atropine (0.0 pmol/mL to 100 pmol/mL) in serum. Standard curves were repeated on 6 separate occasions. Point-by-point coefficients of variability ranged from 9% to 20% for the 13points in the standard curve (mean=16.75%, sem=0.97%). All measurements were performed in triplicate. Data is expressed as percentage inhibition of radioligand binding in pmol/mL atropine equivalents.

### 8. Data Analyses

Curve fitting, estimation of receptor binding parameters and other statistical analyses were performed with GraphPad Prism v. 7 (La Jolla, Calif.). Multiple comparisons following ANOVAs used Sidak-adjusted P levels.

### 9. Binding Parameters in M1WT3 Cells

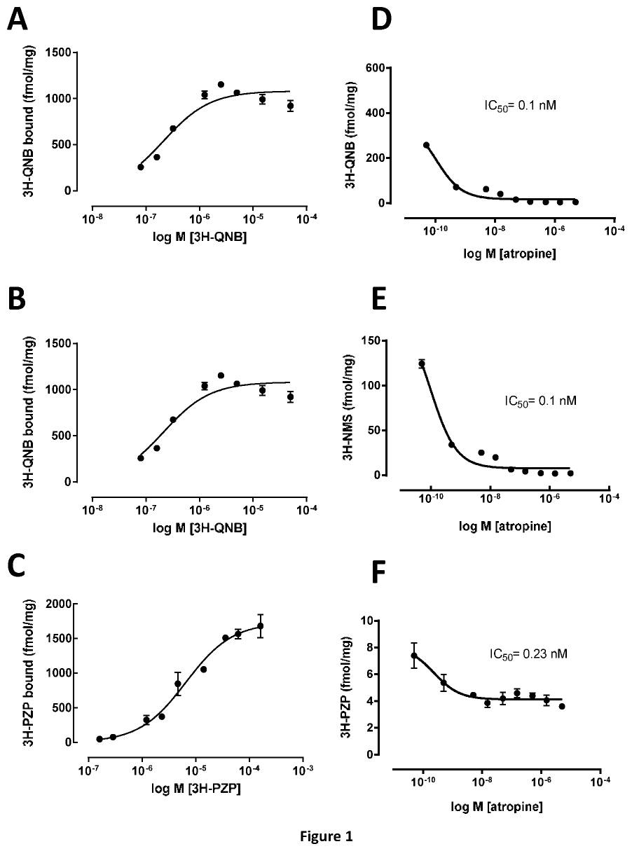

Binding parameters for the standard muscarinic ligand [3H] QNB in cell membranes were determined in buffer. Two other M1 ligands were used for comparison and further validation: [3H] NMS was chosen as a pan-muscarinic receptor ligand similar to [3H] QNB in most respects; [3H] PZP was chosen as an M1-selective ligand, which might potentially provide greater sensitivity. For each of the three M1 ligands, saturation experiments showed that binding to M1WT3 cells was saturable and displaceable by 10 μM atropine (FIG. 1D-F). However, for [3H] QNB and [3H] NMS saturation was achieved at concentrations of 3-15 nM (FIGS. 1A and B), whereas for [3H] PZP it was achieved at concentrations close to 60 nM (FIG. 1C), suggesting that much higher concentrations of [3H] PZP would be needed in comparison to [3H] QNB and [3H] NMS. The estimated Kds for [3H] QNB and [3H] NMS were 0.25 nM and 0.28 nM, respectively, while the estimated Kd for [3H] PZP was 6.76 nM. Atropine showed similar potency to displace equimolar concentrations of [3H] QNB and [3H] NMS (0.16 nM) over a 10⁵-fold concentration range (0.05 nM to 5000 nM)(FIGS. 1D and E), whereas [3H] PZP at the same concentration did not yield a useful signal-to-background ratio (FIG. 1F).

As can be seen in FIG. 2, 0.16 nM [3H] QNB (FIG. 2C) and [3H] NMS (FIG. 2D) showed a similar ability to detect anticholinergic activity in cells using spiked human serum at different dilutions. Non-linear fits for each of the two ligands yielded R2 values of 0.979 for [3H] QNB and 0.988 for [3H] NMS. [3H]QNB values were significantly higher than [3H]NMS values for the four highest concentration points.

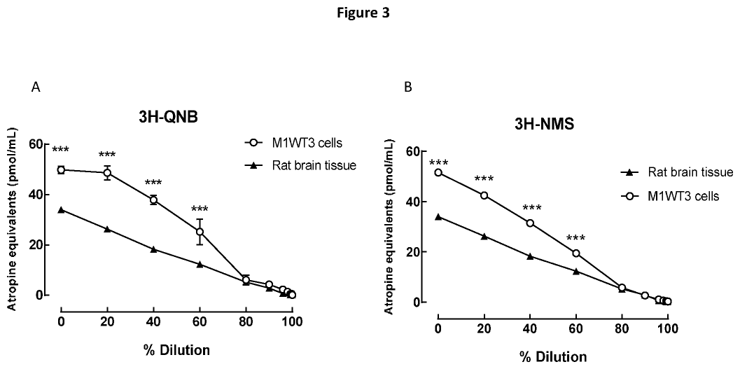

FIG. 3 directly compares the M1WT3 cell assay with the conventional [3H] QNB (FIG. 3A) and [3H] NMS (FIG. 3B) assay using rat brain tissue as a substrate, both assays using the same amount of protein and the same concentration of M1 ligand. As illustrated, significant increases in sensitivity were obtained with the M1WT3 cell assay.

### 11. Effects of Serum Proteins on [3H] QNB and [3H] NMS Binding in M1WT3 Cells

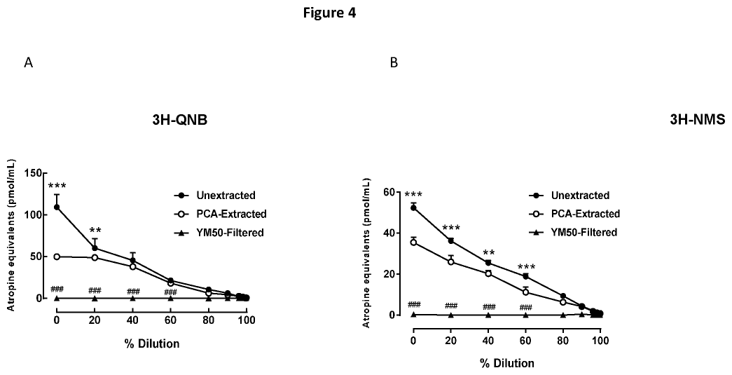

To examine a possible effect of large serum proteins on the cell assay¹³ two different approaches were taken. First, serum filtration experiments were performed by using a 50 kDa membrane filter as previously done by Cox and colleagues and confirmed a substantial loss of binding in the filtered samples, as reported by these authors.¹³ A second approach involved precipitating serum proteins by perchloric acid pre-treatment. Results are shown in FIG. 4. In contrast to filtration, protein precipitation resulted in only a small loss of [3H] QNB binding (FIG. 4A). The same was observed for [3H] NMS (FIG. 4B). BCA protein assays confirmed that in all cases PCA-treated samples contained no measurable amounts of protein.

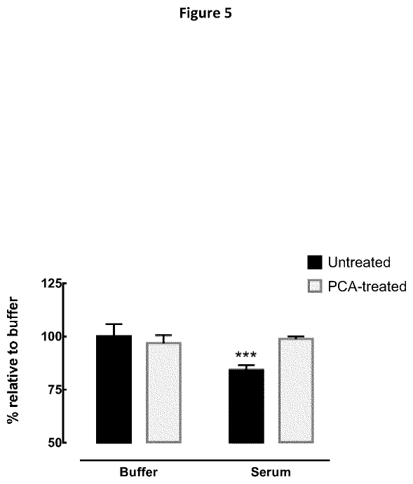

In the course of testing spiked serum it was also noted that normal serum containing no added anticholinergics induced a decrease in binding. This in itself suggested that a component endogenous to normal serum has some ability to decrease binding counts in the absence of any exogenous compounds, possibly by sequestering some of the test radioligand as had been previously suggested.¹³ To address this possibility in the specific case of M1WT3 cells, binding was compared in buffer vs. normal serum containing no known anticholinergic compounds, with or without PCA-treatment. As shown in FIG. 5, binding to M1WT3 cells in normal, unspiked serum was approximately 16% lower than in normal buffer (p<0.0002). Pre-treatment with PCA restored binding to buffer levels (FIG. 5). To verify whether this was a general protein binding effect¹⁶ additional binding experiments were conducted with added bovine serum albumin (BSA) to the samples. Addition of BSA did not affect binding in untreated serum and did not rescue binding losses in PCA-treated samples (data not shown). Likewise, removal of serum lipids¹⁷ had no effect on binding (data not shown).

### Example 2

### Serum Anticholinergic Activity and Cognitive Effects after Acute Scopolamine in Healthy Aged Human Subjects

Assessments of total serum anticholinergic activity (SAA) may be of considerable interest for potential involvement in cognitive impairment associated with, for example, polydrug states in the elderly and other populations. Over the past three decades, SAA assay has been used in efforts to quantify drugs that possess anticholinergic activity in vitro and to document elevated serum anticholinergic levels in community-dwelling and hospitalized patients with delirium and dementia¹⁸⁻²¹. To assess the total burden of anticholinergic activity, a serum anticholinergic activity assay (SAA) was introduced in the early 1980s by Tune and Coyle²²⁻²³ and has since been used as a putative marker of cognitive dysfunction in several conditions, albeit not always with consistent results (for reviews see references 18, 24, and 25). The original Tune and Coyle assay was based on the displacement by human serum of [3H]QNB binding to rat brain homogenates²². Subsequently, however, questions were raised concerning the basic validity of this SAA protocol and several potential limitations have been identified^{18,24}. Among these is a potential role for large serum proteins, which may significantly mask or distort SAA values²⁶. A second potential source of variability in the original SAA protocol is that it did not discriminate between various subtypes of muscarinic receptors, which may limit applicability in studies that are interested in observing the role of anticholinergic burden on cognitive status, as this is a potential predictor of cognitive decline. Only two of the five known muscarinic receptor subtypes (M1 and M2) have been shown to be involved in cognitive functions^{37,38}, where M1 is the most abundant muscarinic receptor in brain³⁹ and the one that has been most clearly implicated in cognitive functions⁴⁰. These and other issues, including the fact that alternatives, such as anticholinergic risk scales, also show lack of uniformity and variability of outcomes⁴¹⁻⁴³ indicate a need for improved, additional, and/or alternative methods for assessing anticholinergic activity.

Furthermore, as discussed above, cholinergic receptor blockade in the central nervous system (CNS) is associated with impaired cognitive function, and for this reason medications with anticholinergic activity are often carefully prescribed and dosed. However, many prescription and non-prescription drugs have varying degrees of anticholinergic activity, and a significant amount of anticholinergic activity may result when these drugs are combined²⁷⁻³². Since CNS cholinergic function diminishes with aging³³, the elderly, who very often take multiple medications for various different types of health issues, are particularly vulnerable in this respect^{20, 30, 34-36}.

As described hereinabove, methods and kits for assessing anticholinergic activity in a sample have now been developed. In Example 1 above, such methods and kits were studied, and used to assess anticholinergic activity in serum samples. In the present Example, methods and kits described herein are used to assess serum anticholinergic activity levels of human serum taken from subjects before and after treatment with scopolamine, a known anticholinergic drug, and treated subjects were further subjected to CANTAB testing to determine cognitive changes following treatment with the scopolamine.

In this Example, subjects were healthy, cognitively intact individuals under well controlled conditions. For this study a sample of 10 individuals were subjected to cognitive tests before and 30 min after an intravenous injection of the cholinergic blocker scopolamine. Serum was likewise collected before and after scopolamine. It was hypothesized that SAA would be increased after single dose scopolamine, and that scores on cognitive measures would correspondingly decline after the treatment.

### Methods ps 1. Cell Culture, Membrane Preparation and 3H-QNB Binding Assay.

a) Chinese hamster ovary (CHO) cells stably expressing rat M1 muscarinic receptors (M1WT3; American Type Culture Collection) were grown in T175 flasks at 37° C. in humidified air and 5% CO₂. Cells were then harvested using 8 mL Accutase solution, rinsed with magnesium- and calcium-free Dulbecco's PBS, and stored at −80° C.

Membranes were prepared according to Lazareno et al. (1998)⁴⁵ with minor modifications. Briefly, cells were homogenized for 30 sec on ice, centrifuged at 40,000×g for 90 min at 4° C., rinsed with 20 mM HEPES, centrifuged again at 40,000×g for 10 min, reconstituted, and stored at −80° C. Protein concentrations were determined using the Pierce BCA Protein Assay Kit.

The binding assay used 100 μL of serum, 27 μg of M1WT3 protein, and 0.16 nM [3H]QNB in HEPES buffer in a total volume of 500 μL, assembled on ice in a 96-well Whatman™ uniplate. The microplate was then incubated at 24° C. for 60 min. Membranes were collected by filtration on GF/B filtermats presoaked in 0.1% PEI, then rinsed with 50 mM Tris HCl, dried, and sealed. Bound radioactivity was counted in a Microbeta-2 microplate scintillation counter.
b) Protein Removal by Precipitation

Prior to their use in the binding assay, serum samples underwent deproteinization, which was performed with a commercial kit (BioVision, Milpitas, Calif.; catalog # K808-200) according to manufacturer's instructions.
c) SAA Assays

Standard curves were constructed for each assay by measuring the displacement of 0.16 nM [3H]QNB binding by atropine (0.0 pmol/mL to 100 pmol/mL) in normal human serum. Standard curves were fitted to a competitive inhibition model which was then used to express test sample anticholinergic activity as inhibition of [3H]QNB binding in pmol/mL atropine equivalents—i.e. the atropine concentration that would induce a comparable reduction in radioligand binding. All measurements were performed in triplicate.

### 2. Clinical Cognitive Measures

Structured Clinical Interview for the DSM-IV (SCID): Participants were screened for any psychiatric disorder including a Neurocognitive Disorder using the Structured Clinical Interview for the DSM IV to determine study eligibility. The SCID-IV assesses current and lifetime depression and other psychiatric disorders. It was used to clarify psychiatric inclusion and exclusion criteria.

The Montreal Cognitive Assessment (MoCA): is a validated, brief cognitive screening tool for detecting mild cognitive impairment (MCI) with high sensitivity and specificity.

Clinical Dementia Rating Scale (CDR): This scale is useful in quantifying the severity of dementia based on six domains of cognitive and functional ability: memory, orientation, judgment and problem solving, community affairs, homes and hobbies, and personal care. Each item is rated on a 5-point scale through a semi-structured interview with the participant or reliable informant.

The 3 tests listed above were used to screen subjects in visit one, to ensure the absence of significant neuropsychiatric impairments. The following tests were administered before and after anticholinergic intervention during visit 2:

### Cambridge Neuropsychological Test Automated Battery (CANTAB-AD):

Two main challenges are often encountered with many of the newer neurocognitive tasks. First is the lack of standardization in administering such tasks. Second is the lack of information on their psychometric properties. The Cambridge Neuropsychological Test Automated Battery (CANTAB) system addresses both of these challenges (www.cambridgecognition.com). The design of the CANTAB is based on well-established neurocognitive experimental paradigms. It was designed initially to provide componential analysis of cognitive functions in the elderly and individuals with dementia. The CANTAB Eclipse consists of 22 tasks that assess neurocognitive processes within a wide range of relatively independent cognitive domains, including visual memory, attention, working memory and problem solving. Each of the tasks is graded, allowing the assessment of patients with varying level of impairment. Increasing the difficulty of a task avoids a ceiling effect. Conversely, decreasing the difficulty of a task avoids a floor effect and allows the distinction between specific and generalized deficits (a concern discussed above). The tasks are automated and therefore testing is given in a standardized manner with a standardized feedback about accuracy and speed. The CANTAB has also a large normative database based on over 2000 normal control subjects, aged 4-90 years. Estimates of test-retest reliability and of practice effects are available for many of the CANTAB tasks based on an elderly sample with an age range of 60-80 years. The CANTAB has been used in a variety of clinical populations providing an opportunity to compare findings related to different disorders, including findings examining cognition in late-life bipolar disorder. Finally, the CANTAB tasks are independent of language and culture and can be used in population for whom English is not a primary language⁴⁶. Administration time is approximately 55 minutes.

### Clinical Samples and Study Medication

Biomarkers: Blood samples were collected for evaluating SAA. Assays will be performed in the laboratory. Total amount of blood to be drawn was four 10 ml blood tubes.

Scopolamine Hydrobromide: Scopolamine is a naturally occurring alkaloid of the belladonna plant. Scopolamine, like atropine, is an antimuscarinic agent antagonizing the action of acetylcholine at muscarinic receptors. The anticholinergic properties of scopolamine and atropine differ in that scopolamine has more pronounced sedative, antisecretory and antiemetic activity while atropine has stronger effects on the heart, intestine and bronchial muscle and a more prolonged duration of action⁴⁷. Scopolamine has many uses including the prevention of motion sickness, treatment of excessive salivation, colicky abdominal pain, sialorrhoea, diverticulitis, irritable bowel syndrome and motion sickness and also postoperative nausea and vomiting⁴⁸.

The variability of absorption and poor bioavailability of oral Scopolamine (Scopace®) indicate that this route of administration may not be reliable and effective⁴⁹ for the purposes of the present assay testing as well as testing the cognitive effects induced by Scopolamine administration. Oral Scopolamine will introduce large variability and may confound any anticipated findings. The advantages of administering it using IV route include rapid onset of action (5-10 minutes), known pharmacokinetics (plasma levels peak at 30 minutes) and shorter half-life (approximately 1 hour)⁴⁷⁻⁵⁰. Another reason is that oral form of Scopolamine is not available in Canada, while the IV form can be supplied by Canadian manufacturers. Accordingly, IV administration of Scopolamine was used.

### Participants and Recruitment

10 cognitively intact healthy participants were recruited. Cognitive assessments were performed on participants who did not meet DSM IV criteria for any diagnosis.

### Study Design

Ten cognitively intact healthy participants underwent cognitive testing prior to and following a single 0.4mg dose of IV scopolamine. Participants attended the Centre for Addiction and Mental Health on two separate occasions:

Visit 1: Study visit 1 was an assessment of eligibility criteria. Information was collected regarding any current or past mental health (Structured Clinical Interview for DSM-IV) or medical issues.

Visit 2: Upon arrival at CAMH, participants were administered the CANTAB. An IV line was then set up by a research nurse and the first blood sample was taken. Following this, the nurse injected 0.4 mg scopolamine. After a 30 minute interval (the time at which scopolamine reaches peak plasma concentration) a second blood sample was drawn. The CANTAB was completed once more. Participants remained on site for observation for 3 hours after scopolamine administration. The qualified investigator or designate was available for the entirety of the study visit.

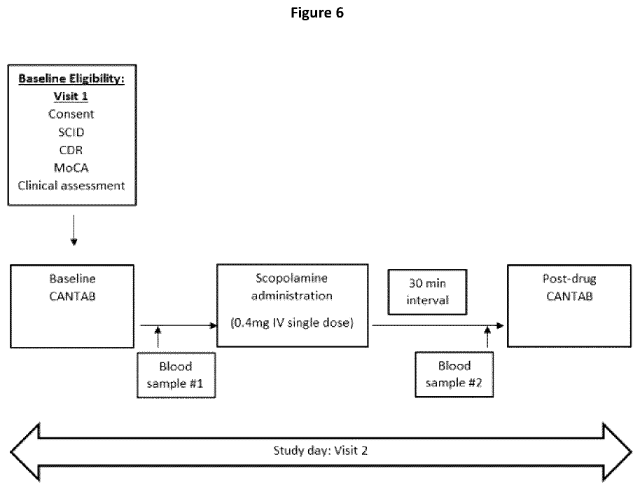

A schematic of the study design is shown in FIG. 6.

### Inclusion/Exclusion Criteria:

No exclusion criteria were based on race, ethnicity, gender, or HIV status.

### Inclusion:

1. Males and females aged 50 or older.
2. Willingness to provide informed consent.
3. Availability of a study partner who has regular contact with the participant to confirm cognitive status.
4. Ability to read and communicate in English (with corrected vision and hearing, if needed)

### Exclusion:

1. Meet any DSM IV criteria for any diagnosis.
2. Significant neurological condition (e.g., stroke, seizure disorder, MS)
3. Unstable medical condition that would preclude safe use of Scopolamine (e.g.

uncontrolled diabetes mellitus, hypertension, tachyarrythmias, glaucoma, benign prostatic hypertrophy, pyloric obstruction, paralytic ileus and myasthenia gravis).
4. Current use of a medication with known potent anticholinergic activity.
5. Hypersensitivity to scopolamine or belladonna alkaloids

### Results

### 1. Subjects

One of the 10 recruited subjects did not return for the second visit and thus 9 of the 10 recruited subjects completed the study. The sample consisted of 2 males and 7 females with ages ranging from 59 to 86 years (mean=69.88, median=71). All subjects were white, non-Hispanic, and on the screening visit did not display evidence of neuropsychiatric deficits on the SCID. They were free of known potent anticholinergic medications and showed no indications of significant cognitive impairment. As shown in Table 2, MoCA scores ranged from 24 to 30 (scores on the MoCA range from 0 to 30, with a score of 26 and higher generally considered normal). For the Clinical Dementia Rating (CDR™) test one subject had a score of 0.5 and all others had a score of zero (Table 2), which is considered normal in this 5-point scale.

### 2. SAA Results

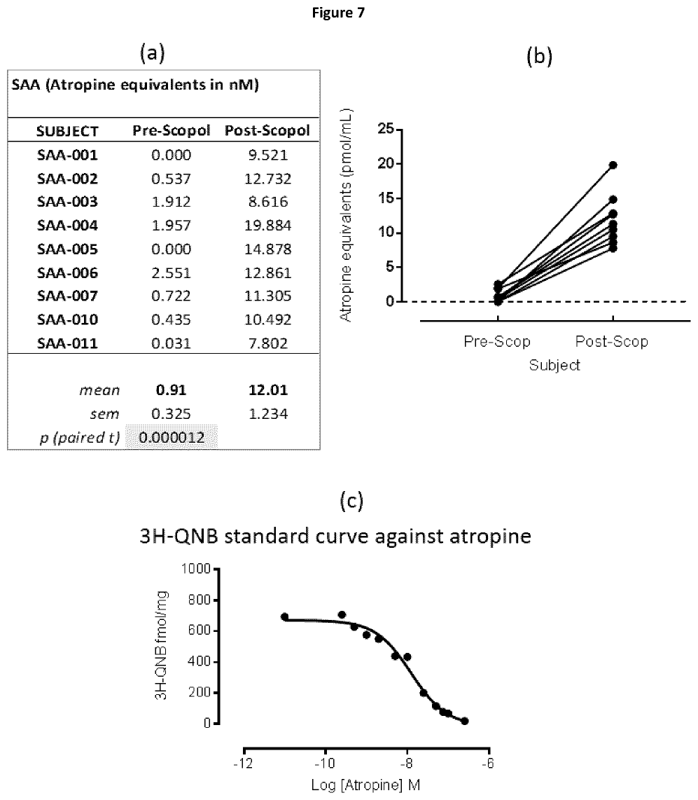

Administration of a single i.v. dose of 0.4 mg scopolamine resulted in a significant increase in SAA activity as measured 30 min later. FIG. 7 shows the individual data for the 9 participants (2 upper panels) as well as the standard curve used to derive atropine equivalents in the cell SAA assay. A paired t test indicated that the difference between pre and post-scopolamine SAA values (0.91±0.32 vs. 12.01±1.234) was highly significant (p<0.000012).

### 3. CANTAB Tests

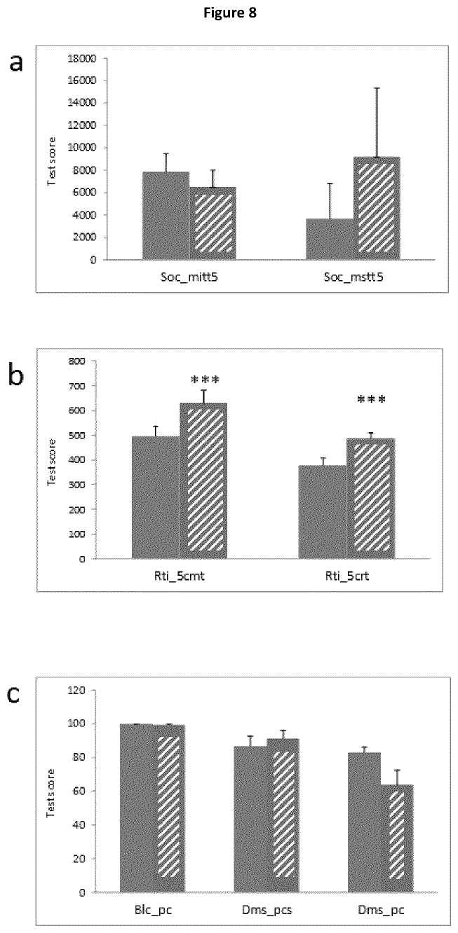

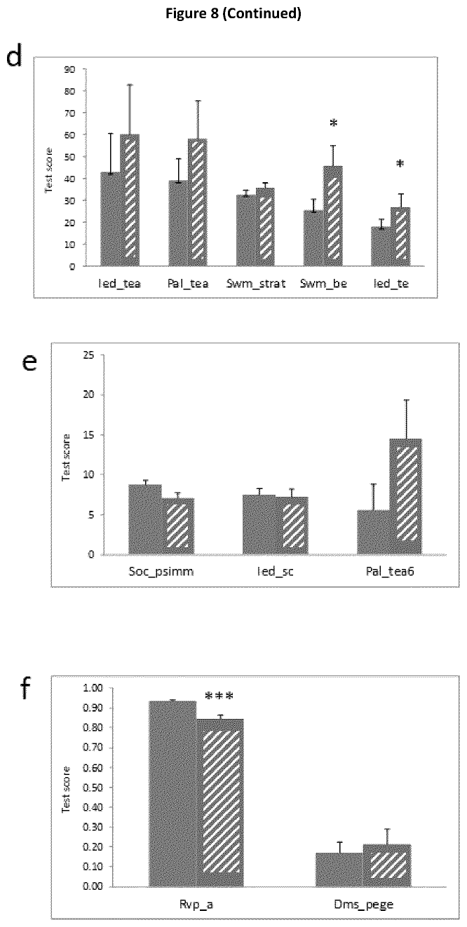

A description of each of the CANTAB variables assessed is provided in Table 1. As shown in FIG. 8 and Table 2, scopolamine resulted in significant increases in a number of cognitive test variables as assessed by various CANTAB tests 30 min after drug administration.

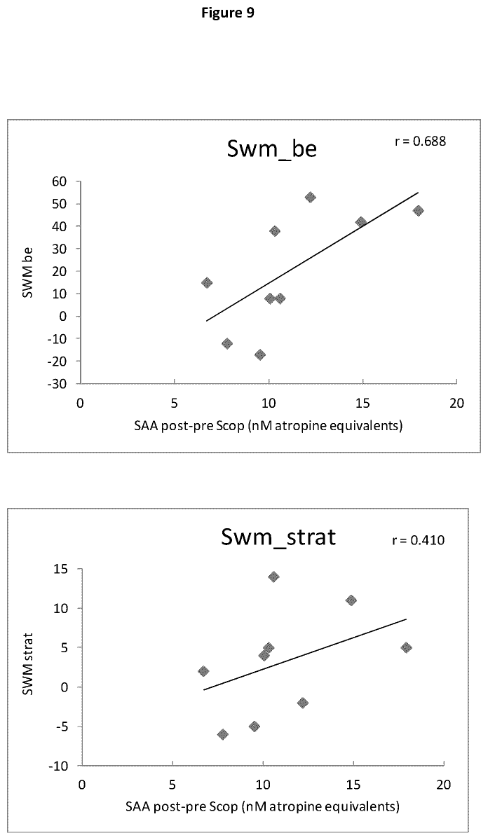

To probe potential associations between SAA changes and changes in cognitive measures, correlations were computed between SAA difference scores (pre-post scopolamine) and CANTAB difference scores (pre-post scopolamine). Despite the low sample size, associations emerged between SAA difference scores and CANTAB difference scores, a difference score referring to pre- vs. post-scopolamine scores in each case. The two highest correlations referred to the positive association between SAA changes and spatial working memory deficits (swm_be, r=0.69, p<0.05) and the positive association between SAA changes and poor use of an appropriate strategy in the spatial working memory test (swm_strat, r=0.41). Scatterplots for these two variables are shown in FIG. 9. It may be of interest that both of these refer to the spatial working memory cognitive domain of CANTAB tests.

### Discussion

The aim of this study was to further investigate the effectiveness of the new cell-based SAA methods described herein in detecting serum anticholinergic activity induced under well-controlled circumstances in a within-subject design. A single i.v. dose of 0.4 mg scopolamine in aged but cognitively intact and drug-free subjects resulted in a strong increase in SAA as measured with the cell assay described above (FIG. 7). This was accompanied by increases in indicators of deficits in various cognitive components indexed by CANTAB test variables. Despite the sample size used, in no case was a failure of scopolamine to induce SAA increases observed in this sample; neither were cases of improvement in cognitive indices observed after scopolamine.

These findings indicate that experimental introduction of a common anticholinergic drug at a dose that is well-within commonly used clinical dosage range led to readily measurable anticholinergic changes using the cell-based assays described herein, which is of particular interest as it demonstrates the effectiveness of the methods and kits described herein for detecting anticholinergic load in a human sample (see FIG. 7, for example).

The fact that the acute treatment also resulted in cognitive changes using well-validated procedures reinforces the importance of accurately assessing anticholinergic load in a number of clinical conditions, particularly in the elderly. The results of this experimental study demonstrates that the presence of anticholinergic drugs in the blood of human subjects may be readily measured by the cell-based assays, methods, and kits described herein.

TABLE 1
Description of CANTAB Variables Tested

Intra-extra dimensional set shift, Stages completed (ied_sc)
This is the total number of stages the subject completed successfully. There are nine stages to be
completed in this task in the clinical mode. Subjects completing all stages are deemed to have
‘passed the test’. There are two key stages, the intra-dimensional shift (stage 6) and the extra-
dimensional shift (stage 8). Analysis of stage reached has often been conducted using the likelihood
ratio method for contingency tables which yields a likelihood ratio statistic ‘2Î’
Intra-extra dimensional set shift, Total errors (ied_te)
This is a measure of the subject's efficiency in attempting the test. Thus, whilst a subject may pass all
nine stages, a substantial number of errors may be made in doing so. It is crucial to note that
subjects failing at any stage of the test have, by definition, had less opportunity to make errors. The
IED Total errors (adjusted) measure attempts to compensate for this.
Intra-extra dimensional set shift, Total errors (adjusted) (ied_tea)
This is a measure of the subject's efficiency in attempting the test. Thus, whilst a subject may pass all
nine stages, a substantial number of errors may be made in doing so. It is crucial to note that
subjects failing at any stage of the test by definition have had less opportunity to make errors.
Therefore, this adjusted score is calculated by adding 25 for each stage not attempted due to failure.
This value of 25 is used since subjects must complete 50 trials to fail a stage and half of these could
be correct by chance alone.
Paired associated learning, Total errors (adjusted) (pal_tea)
This measure reports the total number of errors, with an adjustment for each stage not attempted
due to previous failure. This adjustment is calculated by summing the number of patterns not
attempted and subtracting the number of patterns divided by the number of boxes from it. This
result is then multiplied by the number of trials allowed for the stage (ten in the clinical mode). Note
that for aborted runs, the adjustment is based on the stages, trials and responses not attempted due
to the abort, with each missed response giving rise to an adjustment of 1-1/number of boxes.
Paired associated learning, Total errors (6 shapes, adjusted) (pal_tea6)
This measure reports the total number of errors made at the 6-pattern stage (when there is a
stimulus in each of the 6 boxes), with an adjustment for those who have not reached this stage. This
adjustment is calculated by summing the number of patterns not attempted (6) and subtracting the
number of patterns (6) divided by the number of boxes (6) from it. This result is then multiplied by
the total number of possible trials (10). Thus subjects not reaching this stage are allocated the
number 50. The maximum value for this measure (if the subject makes all possible responses
incorrectly) is 60. The number of errors at the 6-pattern stage of PAL is able to differentiate with 98%
accuracy between patients with Alzheimer's disease and non-demented controls
Reaction Time, Five-choice movement time (rti_5cmt)
This is the time taken to touch the stimulus after the press pad button has been released in trials
where the stimuli has been presented in one of five possible locations. Movement time latency is
measured in milliseconds and is usually normally distributed for correct responses.
Five-choice movement time, taken together with five-choice reaction time, allows us to separate out
any speeding or slowing of motor function from any speeding or slowing of cognitive function
Reaction Time, Five-choice reaction time (rti_5crt)
This is the speed with which the subject releases the press pad button in response to a stimulus in
any one of five locations. Choice reaction time latency is measured in milliseconds and tends toward
a positive skew. Five-choice reaction time latencies are reliably observed to be longer than in simple
reaction time. It should be remembered that subjects engaged in reaction time tasks have the
opportunity to make a variety of errors. Most are errors of commission (‘too soon’, ‘inaccurate’ and
‘wrong circle’), but it is possible to make an error of an omission by not responding (‘too late’).
Latency tasks that contain accuracy demands require a trade-off between speed and accuracy and so
analysis of RT tasks need to consider making reference to both speed and accuracy. Five-choice
reaction time, taken together with five-choice movement time, allows us to separate out any
speeding or slowing of motor function from any speeding or slowing of cognitive function
Rapid image visual processing, A′ (rvp_a)
A′ (A prime) is the signal detection measure of sensitivity to the target, regardless of response
tendency (range 0.00 to 1.00; bad to good). In essence, this measure is a measure of how good the
subject is at detecting target sequences using p(hit) and p(fa). This score is calculated from blocks 5,
6 and 7 only, unless a single block is specified. RVP A′ has been shown to be sensitive to both
neurological damage (such as Alzheimer's disease), and pharmacological manipulation, such as by
the cholinergic agonist, nicotine
Stocking of Cambridge, Mean initial thinking time (5 moves) (soc_mitt5)
Subjects are encouraged to plan their moves before actually enacting the solution to the problems.
Initial thinking time is the difference in time taken to select the first ball for the same problem under
the copy and follow conditions. Therefore, these measures give an indication of the time taken to
plan the problem solution. Possible values for n are 2, 3, 4 or 5. This score may be 0 if the subject is
slower in the ‘follow’ condition. Looking at the initial and subsequent thinking times at the highest
level of difficulty decreases the possibility of ceiling effects and as such, provides a larger potential
for measuring improvements in performance
Stocking of Cambridge, Mean subsequent thinking time (5 moves) (soc_mstt5)
Possible values for n are 2, 3, 4 or 5. These measures reflect the subject's speed of movement after
the initial move has been made for n-move problems. They are obtained by calculating the
difference in time between selecting the first ball and completing the problem for the same problem
under the two conditions (copy and follow), and then dividing this result by the number of moves
made. This score may be 0 if the subject is slower in the ‘follow’ condition. Looking at the initial and
subsequent thinking times at the highest level of difficulty decreases the possibility of ceiling effects
and as such, provides a larger potential for measuring improvements in performance
Stocking of Cambridge, Problems solved in minimum moves (soc_psimm)
This is a fundamental measure, recording the number of occasions upon which the subject has
successfully completed a test problem in the minimum possible number of moves. For the clinical
mode, this is scored out of a possible 12 problems, since eight practice problems are excluded from
the calculation (the first six problems in the first block and the first two problems in the second
block).
This is a succinct expression of overall planning accuracy in SOC. For this measure, you can choose to
apply it to all the assessed problems by not specifying the number of moves in option 1, or you may
use option 1 to specify the number of moves (2, 3, 4 or 5) for the assessed problems for which you
wish to calculate the result.
Spatial working memory, Between errors (swm_be)
Between errors are defined as times the subject revisits a box in which a token has previously been
found. This is calculated for trials of four or more tokens only. This measure is sensitive to
pharmacological manipulation by, for example, diazepam (Coull et al (1995) Psychopharmacology,
120, 311-321), but also sensitive to disorders such as ADHD (Kempton et al (1999) Psychological
Medicine, 29, 527-538).
Spatial working memory, Strategy (swm_strat)
Owen et al. (Neuropsychologia 1990: 28; 1021-1034) have suggested that an efficient strategy for
completing this task is to follow a predetermined sequence by beginning with a specific box and
then, once a blue token has been found, to return to that box to start the new search sequence. An
estimate of the use of this strategy is obtained by counting the number of times the subject begins a
new search with a different box for 6- and 8-box problems only. A high score represents poor use of
this strategy and a low score equates to effective use. Thus, for the clinical mode, the minimum
strategy score is 1 for each stage (i.e. 8) and the maximum is 1 for each search (i.e. 56). It has been
shown to be sensitive to cognitive dysfunction in disorders such as Chronic Fatigue
Big/little circle, Percent correct (blc_pc)
This is the number of correct responses expressed as a percentage of total responses. BLC Percent
correct gives an overall indicator of task performance on Big/Little Circle. This is useful, as some
subjects suffering considerable cognitive difficulties may still be able to attempt Big/Little Circle.
Delayed matching to sample, Percent correct (all delays) (dms_pc)
This measure reports, as a percentage, the number of occasions upon which the subject selected the
correct stimulus in trials when the target stimulus and the three distractors were presented after the
stimulus had been hidden, with delays of 0 ms, 4000 ms and 12000 ms. The percentage of correct
solutions for all delay conditions will give a good overall impression of visual memory ability, when
compared with the percentage of correct solutions for the simultaneous condition. The discrepancy
between percent correct (simultaneous) and percent correct (all delays) indicates the increased
memory load of the delay conditions.
Delayed matching to sample, Percent correct (simultaneous) dms_pcs
This measure reports, as a percentage, the number of occasions upon which the subject selected the
correct stimulus in trials when the stimulus was left in view whilst the target stimulus and the three
distractors were simultaneously presented.
Delayed matching to sample, Prob error given error dms_pege
This measure reports the probability of an error occurring when the previous trial was responded to
incorrectly and is used in calculations of A′ and B″ (A prime and B double prime).

TABLE 2
Cognitive test scores ^{a}

| Screening variables | mean sem | range |
|---|---|---|
| Age | 69.89 ± 3.20 | (59-86) |
| MoCA Total | 27.88 ± 0.88 | (24-30) |
| CDR Total | 0.06 ± 0.06 | (0.0-0.5) |

| Difference scores | | |
|---|---|---|
| CANTAB variables | | |
| Pre vs post scopolamine | | P value ^{b} |
| ied_sc | −0.3 ± 0.6 | 0.594 |
| ied_te | 8.9 ± 3.8 | 0.047 |
| ied_tea | 17.2 ± 13.0 | 0.221 |
| pal_tea | 19.1 ± 18.3 | 0.327 |
| pal_tea6 | 9.0 ± 6.4 | 0.200 |
| rti_5cmt | 132.8 ± 44.4 | 0.017 |
| rti_5crt | 107.7 ± 31.2 | 0.009 |
| rvp_a | −0.1 ± 0.0 | 0.004 |
| soc_mitt5 | −3055.0 ± 1868.0 | 0.153 |
| soc_mstt5 | 5139.3 ± 8163.4 | 0.552 |
| soc_psimm | −1.7 ± 1.0 | 0.125 |
| swm_be | 20.2 ± 8.6 | 0.047 |
| swm_strat | 3.1 ± 2.3 | 0.204 |
| blc_pc | −0.8 ± 0.6 | 0.195 |
| dms_pc | −19.3 ± 8.8 | 0.060 |
| dms_pcs | 4.4 ± 5.6 | 0.447 |
| dms_pege | 0.1 ± 0.1 | 0.439 |

^{a} Values are mean differences post-pre scoplamine ± sem.
See description of CANTAB variables in Table 1
^{b} paired t tests

Various embodiments of compounds, composition and methods for assessing anticholinergic levels in a sample have been described. The above-described embodiments are intended to be examples, and alterations and modifications may be effected thereto by those of ordinary skill in the art without departing from the spirit and scope of the teachings.

All references are herein incorporated by reference in their entireties.

### REFERENCES

1. Tune L, Coyle J T. Serum levels of anticholinergic drugs in treatment of acute extrapyramidal side effects. Arch. Gen. Psychiatry 1980;37:293-7.
2. Tune L, Coyle J T. Acute extrapyramidal side effects: serum levels of neuroleptics and anticholinergics. Psychopharmacology (Berl). 1981;75:9-15.
3. Brecht S, Reiff J, Vock U et al. Serum anticholinergic activity in patients following cardiac surgery and healthy individuals following amitriptyline application. Methods Find. Exp. Clin. Pharmacol. 2007;29:223-30.
4. Carnahan R M, Lund B C, Perry P J et al. A critical appraisal of the utility of the serum anticholinergic activity assay in research and clinical practice. Psychopharmacol. Bull. 2002;36:24-39.
5. Chew M L, Mulsant B H, Pollock B G et al. Anticholinergic activity of 107 medications commonly used by older adults. J. Am. Geriatr. Soc. 2008;56:1333-41.
6. Flacker J M, Lipsitz L A. Serum anticholinergic activity changes with acute illness in elderly medical patients. J. Gerontol. A. Biol. Sci. Med. Sci. 1999;54:M12-6.
7. Hori K, Konishi K, Watanabe K et al. Influence of anticholinergic activity in serum on clinical symptoms of Alzheimer's disease. Neuropsychobiology 2011;63:147-53.
8. Mondimore F M, Damlouji N, Folstein M F et al. Post-ECT confusional states associated with elevated serum anticholinergic levels. Am. J. Psychiatry 1983;140:930-1.
9. Nebes R D, Pollock B G, Halligan E M et al. Cognitive slowing associated with elevated serum anticholinergic activity in older individuals is decreased by caffeine use. Am. J. Geriatr. Psychiatry 2011;19:169-75.
10. Plaschke K, Hill H, Engelhardt R et al. EEG changes and serum anticholinergic activity measured in patients with delirium in the intensive care unit. Anaesthesia 2007;62:1217-23.
11. Thienhaus O J, Allen A, Bennett J A et al. Anticholinergic serum levels and cognitive performance. Eur. Arch. Psychiatry Clin. Neurosci. 1990;240:28-33.
12. Tune L, Carr S, Hoag E et al. Anticholinergic effects of drugs commonly prescribed for the elderly: potential means for assessing risk of delirium. Am. J. Psychiatry 1992;149:1393-4.
Cox E A, Kwatra S G, Shetty S et al. Flaws in the serum anticholinergic activity assay: implications for the study of delirium. J. Am. Geriatr. Soc. 2009;57:1707-8.
14. Kersten H, Wyller T B. Anticholinergic drug burden in older people's brain—how well is it measured? Basic Clin Pharmacol Toxicol 2014;114:151-9.
15. Lazareno S, Gharagozloo P, Kuonen D et al. Subtype-selective positive cooperative interactions between brucine analogues and acetylcholine at muscarinic receptors: radioligand binding studies. Mol. Pharmacol. 1998;53:573-89.
16. Fasano M, Curry S, Terreno E et al. The extraordinary ligand binding properties of human serum albumin. IUBMB life 2005;57:787-96.
17. Castro A R, Morrill W E, Pope V. Lipid removal from human serum samples. Clin. Diagn. Lab. Immunol. 2000;7:197-9.
18. Carnahan R M, Lund B C, Perry P J, Pollock B G. A critical appraisal of the utility of the serum anticholinergic activity assay in research and clinical practice. Psychopharmacol Bull Spring 2002;36(2):24-39.
19. Boustani M, Campbell N, Munger S, Maidment I, Fox C. Impact of anticholinergics on the aging brain: a review and practical application. Aging Health 2008, Jun. 1, 2008;4(3):311-320.
20. Chew M L, Mulsant B H, Pollock B G, et al. Anticholinergic activity of 107 medications commonly used by older adults. J Am Geriatr Soc Jul 2008;56(7):1333-1341.
21. Ruxton K, Woodman R J, Mangoni A A. Drugs with anticholinergic effects and cognitive impairment, falls and all-cause mortality in older adults: A systematic review and meta-analysis. Br J Clin Pharmacol August 2015;80(2):209-220.
22. Tune L, Coyle J T. Serum levels of anticholinergic drugs in treatment of acute extrapyramidal side effects. Arch Gen Psychiatry March 1980;37(3):293-297.
23. Tune L, Coyle J T. Acute extrapyramidal side effects: serum levels of neuroleptics and anticholinergics. Psychopharmacology (Berl) 1981;75(1):9-15.
24. Salahudeen M S, Chyou T Y, Nishtala P S. Serum anticholinergic activity and cognitive and functional adverse outcomes in older people: A systematic review and meta-analysis of the literature. PloS one 2016;11(3):e0151084.
25. Staskin D R, Zoltan E. Anticholinergics and central nervous system effects: are we confused? Reviews in urology Fall 2007;9(4):191-196.
26. Cox E A, Kwatra S G, Shetty S, Kwatra M M. Flaws in the serum anticholinergic activity assay: implications for the study of delirium. J Am Geriatr Soc September 2009;57(9):1707-1708.
27. Campbell N, Boustani M, Limbil T, et al. The cognitive impact of anticholinergics: a clinical review. Clinical interventions in aging 2009;4:225-233.
28. Carnahan R M, Lund B C, Perry P J, Pollock B G, Culp K R. The Anticholinergic Drug Scale as a measure of drug-related anticholinergic burden: associations with serum anticholinergic activity. J Clin Pharmacol December 2006;46(12):1481-1486.
29. Chew M L, Mulsant B H, Pollock B G. Serum anticholinergic activity and cognition in patients with moderate-to-severe dementia. Am J Geriatr Psychiatry June 2005;13(6):535-538.
30. Lampela P, Lavikainen P, Garcia-Horsman J A, Bell J S, Huupponen R, Hartikainen S. Anticholinergic drug use, serum anticholinergic activity, and adverse drug events among older people: a population-based study. Drugs Aging May 2013;30(5):321-330.
31. Cardwell K, Hughes C M, Ryan C. The association between anticholinergic medication burden and health related outcomes in the ‘Oldest Old’: A systematic review of the literature. Drugs Aging Oct 2015;32(10):835-848.
32. Gerretsen P, Pollock B G. Drugs with anticholinergic properties: a current perspective on use and safety. Expert opinion on drug safety September 2011;10(5):751-765.
33. Cai X, Campbell N, Khan B, Callahan C, Boustani M. Long-term anticholinergic use and the aging brain. Alzheimers Dement July 2013;9(4):377-385.
34. Gnjidic D, Le Couteur D G, Naganathan V, et al. Effects of drug burden index on cognitive function in older men. J Clin Psychopharmacol April 2012;32(2):273-277.
35. Flacker J M, Cummings V, Mach J R, Jr., Bettin K, Kiely D K, Wei J. The association of serum anticholinergic activity with delirium in elderly medical patients. Am J Geriatr Psychiatry Winter 1998;6(1):31-41.
36. Flacker J M, Lipsitz L A. Serum anticholinergic activity changes with acute illness in elderly medical patients. J Gerontol A Biol Sci Med Sci January 1999;54(1):M12-16.
37. Anagnostaras S G, Murphy G G, Hamilton S E, Mitchell S L, Rahnama N P, Nathanson N M, Silva A J. Selective cognitive dysfunction in acetylcholine M1 muscarinic receptor mutant mice. Nat Neurosci January 2003;6(1):51-58.
38. Messer W S, Jr., Bohnett M, Stibbe J. Evidence for a preferential involvement of M1 muscarinic receptors in representational memory. Neurosci Lett Aug. 14, 1990;116(1-2):184-189.
39. Levey A l, Kitt C A, Simonds W F, Price D L, Brann M R. Identification and localization of muscarinic acetylcholine receptor proteins in brain with subtype-specific antibodies. J Neurosci October 1991;11(10):3218-3226.
40. Abrams P, Andersson K E, Buccafusco J J, et al. Muscarinic receptors: their distribution and function in body systems, and the implications for treating overactive bladder. Br J Pharmacol July 2006;148(5):565-578.
41. Salahudeen M S, Duffull S B, Nishtala P S. Anticholinergic burden quantified by anticholinergic risk scales and adverse outcomes in older people: a systematic review. BMC geriatrics Mar. 25, 2015;15:31.
42. Villalba-Moreno A M, Alfaro-Lara E R, Perez-Guerrero M C, Nieto-Martin M D, Santos-Ramos B. Systematic review on the use of anticholinergic scales in poly pathological patients. Arch Gerontol Geriatr January-February 2016;62:1-8.
43. Kersten H, Wyller T B. Anticholinergic drug burden in older people's brain—how well is it measured? Basic Clin Pharmacol Toxicol February 2014;114(2):151-159.
44. Chew M L, Mulsant B H, Pollock B G, et al. Anticholinergic activity of 107 medications commonly used by older adults. Journal of the American Geriatrics Society July 2008;56(7):1333-1341.
45. Lazareno S, Gharagozloo P, Kuonen D, Popham A, Birdsall N J. Subtype-selective positive cooperative interactions between brucine analogues and acetylcholine at muscarinic receptors: radioligand binding studies. Mol Pharmacol March 1998;53(3):573-589.
46. Strauss E, Sherman E M S, Spreen O. A Compendium of Neuropsychological Tests: Administration, Norms, and Commentary: Oxford University Press; 2006.
47. Canadian Pharmacists Association. Compendium of Therapeutic Choices, CTC 7. Vol xxv. Seventh edition ed; 2014.
48. (NLM) NLoM. TOXNET®: Toxicology Data Network. Available at: https://toxnet.nlm.nih.gov/.
49. Putcha L, Cintron N M, Tsui J, Vanderploeg J M, Kramer W G. Pharmacokinetics and oral bioavailability of scopolamine in normal subjects. Pharm Res June 1989;6(6):481-485.
50. Renner U D, Oertel R, Kirch W. Pharmacokinetics and pharmacodynamics in clinical use of scopolamine. Ther Drug Monit October 2005;27(5):655-665.
51. Lenz R A, Baker J D, Locke C, Rueter L E, Mahler E G, Wesnes K, Abi-Saab W, Saltarelli M D. The scopolamine model as a pharmacodynamic marker in early drug development. Psychopharmacology 2012;220(1):97-107.
52. Snyder P J, Bednar M M, Cromer J R, Maruff P. Reversal of scopolamine-induced deficits with a single dose of donepezil, an acetylcholinesterase inhibitor. Alzheimer's & Dementia: The Journal of the Alzheimer's Association;1(2):126-135.
53. Nebes R D, Pollock B G, Halligan E M, Houck P, Saxton J A. Cognitive Slowing Associated With Elevated Serum Anticholinergic Activity in Older Individuals is Decreased by Caffeine Use. The American Journal of Geriatric Psychiatry 2//2011;19(2):169-175.
54. Klinkenberg I, Blokland A. The validity of scopolamine as a pharmacological model for cognitive impairment: a review of animal behavioral studies. Neurosci Biobehav Rev July 2010;34(8):1307-1350.
55. Broks P, Preston G C, Traub M, Poppleton P, Ward C, Stahl S M. Modelling dementia: effects of scopolamine on memory and attention. Neuropsychologia 1988;26(5):685-700.
56. Fredrickson A, Snyder P J, Cromer J, Thomas E, Lewis M, Maruff P. The use of effect sizes to characterize the nature of cognitive change in psychopharmacological studies: an example with scopolamine. Human Psychopharmacology: Clinical and Experimental 2008;23(5):425-436.
57. Petersen R. Scopolamine induced learning failures in man. Psychopharmacology 1977;52(3):283-289.
58. Ebert U, Siepmann M, Oertel R, Wesnes K A, Kirch W. Pharmacokinetics and Pharmacodynamics of Scopolamine after Subcutaneous Administration. The Journal of Clinical Pharmacology 1998;38(8):720-726.
59. Canadian Ophthalmological Society evidence-based clinical practice guidelines for the management of glaucoma in the adult eye. Can J Ophthalmol 2009;44 Suppl 1:S7-93.
60. Canada CoFPo. Screening for open angle glaucoma? A guideline to detecting and referring glaucoma suspects for family physicians. 2014.
61. Gupta D, Chen P P. Glaucoma. Am Fam Physician Apr. 15, 2016;93(8):668-674.
62. Prum B E, Jr., Herndon L W, Jr., Moroi S E, et al. Primary Angle Closure Preferred Practice Pattern((R)) Guidelines. Ophthalmology January 2016;123(1):P1-p40.

All references are herein incorporated by reference in their entireties.

What is claimed is:

1. A method for determining the level of muscarinic acetylcholine receptor subtype-1 (M1 receptor) anticholinergic activity in a blood serum sample, the method comprising:
removing protein from the blood serum sample by treatment with perchloric acid (PCA) to produce a PCA-treated serum sample;
incubating the PCA-treated serum sample with a membrane preparation from cultured cells expressing the M1 receptor and an M1 receptor ligand;
detecting an amount of binding of the M1 receptor ligand to the M1 receptor and comparing the amount of binding to a standard to determine the level of M1 receptor anticholinergic activity in the blood serum sample.

2. The method of claim 1, wherein the M1 receptor ligand is [3H] quinuclidinyl benzilate (3H-QNB), [3H] N-methyl-scopolamine (3H-NMS) or [3H] pirenzepine (3H-PZP).

3. The method of claim 2, wherein the M1 receptor ligand is 3H-QNB or 3H-NMS.

4. The method of claim 1, wherein the blood serum sample is derived from a patient or subject that exhibits one or more signs or symptoms of high or elevated M1 receptor anticholinergic activity, is suspected of having high or elevated blood levels of M1 receptor anticholinergic activity, exhibits no symptoms of high M1 receptor anticholinergic activity, or wherein the level of anticholinergic activity is unknown.

5. The method of claim 1 wherein the standard is atropine.

6. The method of claim 5 wherein binding of atropine to the M1 receptor is performed to generate one or more standard curves.

7. The method of claim 5 wherein binding of atropine to the M1 receptor is performed under essentially the same conditions as the M1 receptor binding to the M1 ligand.

8. The method of claim 5 wherein the level of M1 receptor anticholinergic activity is expressed as atropine equivalents.

9. The method of claim 4, wherein a high or elevated M1 receptor anticholinergic activity is equivalent to or higher than 20, 40, 60, 80, 100, 120, or 140 pmol/mL atropine, and optionally associated with an age, a minimum age or an age range.

10. The method of claim 1, wherein the standard is atropine and the level of M1 receptor anticholinergic activity in the blood serum sample is calculated on the basis of the amount of atropine that would provide a substantially similar or identical degree of inhibition of the specific binding of the M1 receptor ligand to the M1 receptor.

11. The method of claim 4, wherein one or more signs or symptoms comprise one or more cognitive side effects or non-cognitive side effects such as, but not limited to dementia, memory loss, cognitive decline, decrease in global cognitive functioning, psychomotor speed, decrease in visual and/or declarative memory, implicit learning or communication ability, confusion, disorientation, agitation, euphoria or dysphoria, respiratory depression, inability to concentrate, inability to sustain a train of thought, incoherent speech, irritability, wakeful myoclonic jerking, unusual sensitivity to sudden sounds, illogical thinking, photophobia, visual disturbances, visual, auditory, or other sensory hallucinations, orthostatic hypotension, urinary problems and/or kidney failure, salivary problems such as dry mouth, blurred vision, constipation, hypohydrosis, dizziness and the like.

12. The method of claim 1, wherein the M1 receptor is a human M1 receptor or rat M1 receptor.

13. The method of claim 1, wherein the ratio of the membrane preparation:treated serum sample is about 0.27 g:1 L and the M1 ligand is employed in the amount of about 0.16 nM.

14. The method of claim 1, wherein binding employs a buffer comprising 20 mM HEPES, 100 mM NaCl and 10 mM MgCl₂ adjusted to a pH of 7.4.

15. The method of claim 1, wherein the PCA-treated serum sample, membrane preparation, M1 ligand and buffer are mixed at about 0° C. and the incubating step is performed at about 24° C. for about 60-120 min.

16. The method of claim 1, wherein after the step of incubating and before the step of detecting, an unbound M1 ligand is removed by filtering the membrane preparation on a filter with a pore size suitable for filtering unbound M1 ligand and retaining the M1 receptor followed by rinsing the membrane preparation.

17. The method of claim 1, performed in a multiwell plate.

18. A method for assessing anticholinergic activity of a sample, the method comprising:
obtaining a membrane preparation from cultured cells expressing rat muscarinic receptor subtype 1 (M1 receptor);
incubating the treated serum sample with the membrane preparation and an M1 ligand;
detecting an amount of binding of the M1 ligand to the M1 receptor and comparing the amount of binding to a standard.

19. A method for assessing anticholinergic activity of a serum sample, the method comprising:
removing protein from the serum sample by treatment with perchloric acid to produce a treated serum sample;
culturing cells expressing rat muscarinic receptor subtype 1 (M1 receptor);
obtaining a membrane preparation from the cells;
incubating the treated serum sample with the membrane preparation and an M1 ligand, wherein the M1 ligand is 3H-QNB or 3H-NMS;
removing unbound M1 ligand;
detecting an amount of binding of the M1 ligand to the M1 receptor and comparing the amount of binding to a standard.

20. A method of modulating serum anticholinergic activity in a patient or subject receiving medication, the method comprising:
obtaining a serum sample from the patient;
removing protein from the serum sample by treatment with perchloric acid to produce a treated serum sample;
obtaining a membrane preparation from cultured cells expressing rat muscarinic receptor subtype 1 (M1 receptor);
incubating the treated serum sample with the membrane preparation and an M1 ligand;
detecting an amount of binding of the M1 ligand to the M1 receptor and comparing the amount of binding to a standard; and
modulating the dosage and/or type of the medication.

21. The method of claim 20, wherein the method further comprises repeating the steps before the modulating step to determine if the change in the dosage and/or type of the medication modulated the serum anticholinergic activity.

22. A method of modulating serum anticholinergic activity of a patient receiving medication and exhibiting one or more signs of cognitive side effects, the method comprising:
obtaining a serum sample from the patient;
removing protein from the serum sample by treatment with perchloric acid to produce a treated serum sample;
obtaining a membrane preparation from cultured cells expressing rat muscarinic receptor subtype 1 (M1 receptor);
incubating the treated serum sample with the membrane preparation and an M1 ligand;
detecting an amount of binding of the M1 ligand to the M1 receptor and comparing the amount of binding to a standard; and
modulating the dosage and/or type of the medication to decrease the serum anticholinergic activity.

23. The method of claim 22, wherein the method further comprises repeating the steps before the modulating step to determine if the change in the dosage and/or type of the medication decreased the serum anticholinergic activity.

24. A kit for assessing or determining anticholinergic activity comprising one or more of the following components in any combination: cells expressing an M1 receptor, one or more cell culture media, one or more cell wash media, one or more buffers, protein concentration assay determination reagent(s), one or more anticholinergic compounds or compositions, atropine, one or more multiwell plates, M1 membrane preparations adhered to a plate or other substrate, one or more filtration membranes, scintillation fluid, one or more M1 ligands, deproteinization solution, perchloric acid, perchloric acid neutralization solution, data analysis software, serum containing one or more anticholinergic compounds or compositions, glassware, centrifuge tubes, instructions for performing an anticholinergic assay or any combination thereof.

25. A kit for assessing anticholinergic activity of a serum sample, the kit comprising:
perchloric acid to remove protein from the serum sample to produce a treated serum sample;
perchloric acid neutralizing solution;
a membrane preparation from cultured cells expressing rat muscarinic receptor subtype 1 (M1 receptor); and
an M1 ligand.

26. The kit of claim 25, wherein the kit further comprises one or more multiwell plates, or one or more multi-well plates comprising a filter with a pore size capable of filtering unbound M1 ligand and retaining the M1 receptor, and buffer.

27. The kit of claim 26, wherein the kit further comprises scintillation fluid.

28. A kit for assessing anticholinergic activity of a serum sample, the kit comprising:
perchloric acid to remove protein from the serum sample to produce a treated serum sample;
perchloric acid neutralizing solution;
an M1 ligand;
buffer;
one or more multi-well plates comprising, in each well, a membrane preparation from cultured cells expressing rat muscarinic receptor subtype 1 (M1 receptor), or one or more multi-well plates comprising a filter with a pore size capable of filtering unbound M1 ligand and retaining the M1 receptor, both or a combination thereof.

29. The kit of claim 28, wherein the kit further comprises scintillation fluid.

30. A method for identifying a subject as being at risk of having or developing cognitive impairment, the method comprising:
providing a blood serum sample from the subject;
removing protein from the blood serum sample by treatment with perchloric acid (PCA) to produce a PCA-treated serum sample;
incubating the PCA-treated serum sample with a membrane preparation from cultured cells expressing the M1 receptor and an M1 receptor ligand; and
detecting an amount of binding of the M1 receptor ligand to the M1 receptor and comparing the amount of binding to a standard to determine a level of muscarinic acetylcholine receptor subtype-1 (M1 receptor) anticholinergic activity in the blood serum sample;
wherein an elevated level of M1 anticholinergic activity in the blood serum sample as compared to a healthy control level identifies the subject as being at risk of having or developing cognitive impairment.

31. The method of claim 30, further comprising a step of:
subjecting the subject identified as being at risk of having or developing cognitive impairment to a Cambridge Neuropsychological Test Automated Battery (CANTAB-AD) to further assess cognitive impairment of the subject.

32. The method of claim 30 or 31, wherein the subject is a subject being treated with at least one drug having anticholinergic properties.

33. The method of any one of claims 30-32, wherein the cognitive impairment is in the spatial working memory cognitive domain.
